(19) United States
(12) Patent Application Publication
(10) Pub. No.: US 2026/0182850 A1
(43) Pub. Date: Jul. 2, 2026

(54) METHOD AND SYSTEM FOR DETERMINING BLOOD FLOW PARAMETER OF RETICULAR VESSEL

(71) Applicant: SHANGHAI UNITED IMAGING HEALTHCARE CO., LTD., Shanghai (CN)

(72) Inventor: Jian GUO, Shanghai (CN)

(73) Assignee: SHANGHAI UNITED IMAGING HEALTHCARE CO., LTD., Shanghai (CN)

(21) Appl. No.: 19/549,085

(22) Filed: Feb. 25, 2026

Related U.S. Application Data
(63) Continuation of application No. PCT/CN2024/114394, filed on Aug. 25, 2024.

(30) Foreign Application Priority Data
Aug. 25, 2023 (CN) 202311085677.5

(51) Int. Cl. A61B 5/026; G06T 7/00

(52) U.S. Cl. CPC A61B 5/0261; G06T 7/0012; G06T 2207/20081; G06T 2207/20112; G06T 2207/30104

(57) ABSTRACT

Embodiments of the present disclosure provide a method and a system for determining a blood flow parameter of a reticular vessel. The method may be implemented on a device including at least one processing device and at least one storage device. The method may include: obtaining vessel data of an object; generating a reticular vessel model by performing, based on the vessel data, vessel segmentation; generating, by performing model partitioning based on the reticular vessel model, a plurality of vessel sub-models; and determine the blood flow parameter of the reticular vessel model by performing, based on the plurality of vessel sub-models, coupling processing.

### CROSS-REFERENCE TO RELATED APPLICATION

This application is a continuation of International Application No. PCT/CN2024/114394, filed on Aug. 25, 2024, which claims priority of Chinese Patent Application No. 202311085677.5, filed on Aug. 25, 2023, the entire contents of which are incorporated herein by reference.

### TECHNICAL FIELD

The present disclosure relates to the field of medical technology, and in particular, to a method and a system for determining a blood flow parameter of a reticular vessel.

### BACKGROUND

Reticular vessels have a significant impact on the life and health of patients. For example, when the reticular vessel is an intracranial blood vessel, lesions of intracranial blood vessel include aneurysms and arterial stenosis. However, there is a lack of effective clinical indicators for assessing the intracranial vessel lesions, and this situation also occurs in other reticular vessels.

Therefore, some embodiments of the present disclosure provide a method and a system for determining a blood flow parameter of a reticular vessel to obtain effective blood flow parameters of the reticular vessels for assessment of intracranial vessel lesions.

### SUMMARY

One or more embodiments of the present disclosure may provide a method for determining a blood flow parameter of a reticular vessel. The method may be implemented on a device including at least one processor and at least one storage device. The method may include: obtaining vessel data of an object; generating a reticular vessel model by performing, based on the vessel data, vessel segmentation; generating, by performing model partitioning based on the reticular vessel model, a plurality of vessel sub-models; and determining the blood flow parameter of the reticular vessel model by performing, based on the plurality of vessel sub-models, coupling processing. In some embodiments, the method may include: obtaining a vessel image of an object; generating a reticular vessel model by performing, based on the vessel image, vessel segmentation; generating, by performing model partitioning based on the reticular vessel model, a plurality of vessel sub-models; and determining the blood flow parameter of the reticular vessel model by performing, based on the plurality of vessel sub-models, coupling processing.

One or more embodiments of the present disclosure may provide a system for determining a blood flow parameter of a reticular vessel, including: a data acquisition module configured to obtain vessel data of an object; a vessel model generation module configured to generate a reticular vessel model by performing, based on the vessel data, vessel segmentation; a model partitioning module configured to generate, by performing model partitioning based on the reticular vessel model, a plurality of vessel sub-models; and a coupling processing module configure to determine the blood flow parameter of the reticular vessel model by performing, based on the plurality of vessel sub-models, coupling processing. In some embodiments, the system may include: a data acquisition module configured to obtain a vessel image of an object; a vessel model generation module configured to generate a reticular vessel model by performing, based on the vessel image, vessel segmentation; a model partitioning module configured to generate, by performing model partitioning based on the reticular vessel model, a plurality of vessel sub-models; and a coupling processing module configure to determine the blood flow parameter of the reticular vessel model by performing, based on the plurality of vessel sub-models, coupling processing.

One or more embodiments of the present disclosure may provide a device for determining a blood flow parameter of a reticular vessel including at least one processor. The processor may be configured to perform operations including: obtaining vessel data of an object; generating a reticular vessel model by performing, based on the vessel data, vessel segmentation; generating, by performing model partitioning based on the reticular vessel model, a plurality of vessel sub-models; and determining the blood flow parameter of the reticular vessel model by performing, based on the plurality of vessel sub-models, coupling processing. In some embodiments, the processor may be configured to perform operations including: obtaining a vessel image of an object; generating a reticular vessel model by performing, based on the vessel image, vessel segmentation; generating, by performing model partitioning based on the reticular vessel model, a plurality of vessel sub-models; and determining the blood flow parameter of the reticular vessel model by performing, based on the plurality of vessel sub-models, coupling processing.

One or more embodiments of the present disclosure may provide a non-transitory computer readable storage medium including at least one set of instructions. When executed by one or more processors of a computing device, the at least one set of instructions causes the computing device to perform a method. The method may include: obtaining vessel data of an object; generating a reticular vessel model by performing, based on the vessel data, vessel segmentation; generating, by performing model partitioning based on the reticular vessel model, a plurality of vessel sub-models; and determining the blood flow parameter of the reticular vessel model by performing, based on the plurality of vessel sub-models, coupling processing. In some embodiments, the method may include: obtaining a vessel image of an object; generating a reticular vessel model by performing, based on the vessel image, vessel segmentation; generating, by performing model partitioning based on the reticular vessel model, a plurality of vessel sub-models; and determining the blood flow parameter of the reticular vessel model by performing, based on the plurality of vessel sub-models, coupling processing.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an application scenario of a system for determining a blood flow parameter of a reticular vessel according to some embodiments of the present disclosure;

FIG. 2 is an exemplary flowchart illustrating an exemplary process for determining a blood flow parameter of a reticular vessel according to some embodiments of the present disclosure;

FIG. 3 is an exemplary flowchart illustrating an exemplary process for partitioning according to some embodiments of the present disclosure;

FIG. 4 is an exemplary flowchart illustrating an exemplary process for coupling processing according to some embodiments of the present disclosure;

FIG. 5 is an exemplary flowchart illustrating an exemplary process for coupling processing according to some other embodiments of the present disclosure;

FIG. 6 is an exemplary block diagram illustrating an exemplary system for determining a blood flow parameter of a reticular vessel according to some embodiments of the present disclosure;

FIG. 7 is an exemplary schematic diagram illustrating an exemplary process for segmenting a ring sub-model according to some embodiments of the present disclosure;

FIG. 8 is an exemplary schematic diagram illustrating an exemplary process for segmenting an H-shaped model according to some embodiments of the present disclosure;

FIG. 9 is another exemplary schematic diagram illustrating an exemplary process for segmenting an H-shaped model according to some embodiments of the present disclosure;

FIG. 10 is an exemplary schematic diagram illustrating an exemplary relationship of a pre-order and a post-order of a sub-model according to some embodiments of the present disclosure;

FIG. 11 is an exemplary schematic diagram illustrating an exemplary structure of a Willis ring according to some embodiments of the present disclosure; and

FIG. 12 is an exemplary schematic diagram illustrating an exemplary collision flow according to some embodiments of the present disclosure.

### DETAILED DESCRIPTION

In the following detailed description, numerous specific details are set forth by way of examples in order to provide a thorough understanding of the relevant disclosure. Obviously, drawings described below are only some examples or embodiments of the present disclosure. Those skilled in the art, without further creative efforts, may apply the present disclosure to other similar scenarios according to these drawings. It should be understood that the purposes of these illustrated embodiments are only provided to those skilled in the art to practice the application, and not intended to limit the scope of the present disclosure. Unless obviously obtained from the context or the context illustrates otherwise, the same numeral in the drawings refers to the same structure or operation.

It will be understood that the terms “system,” “device,” and/or “module” used herein are one method to distinguish different components, elements, parts, sections, or assemblies of different levels in ascending order. However, the terms may be displaced by other expressions if they may achieve the same purpose.

The terminology used herein is for the purposes of describing particular examples and embodiments only and is not intended to be limiting. As used herein, the singular forms “a,” “an,” and “the” may be intended to include the plural forms as well, unless the context clearly indicates otherwise. It will be further understood that the terms “include” and/or “comprise,” when used in this disclosure, specify the presence of integers, devices, behaviors, stated features, operations, elements, operations, and/or components, but do not exclude the presence or addition of one or more other integers, devices, behaviors, features, operations, elements, operations, components, and/or groups thereof.

The flowcharts used in the present disclosure illustrate operations that systems implement according to some embodiments of the present disclosure. It is to be expressly understood, the operations of the flowcharts may be implemented not in order. Conversely, the operations may be implemented in an inverted order, or simultaneously. Moreover, one or more other operations may be added to the flowcharts. One or more operations may be removed from the flowcharts.

As used herein, a reticular vessel model refers to a reticular vessel structure in which there may be one or more nodes without a parent node, and one child node is allowed to be connected to more than one parent node at a same time. The nodes represent branches or intersections in the reticular vessel model, which may include one or more parent nodes and child nodes. A child node is a node that is located downstream of a parent node and is directly connected to the parent node.

The reticular vessel described in the present disclosure may be in different parts of an object. In some embodiments, the reticular vessel may be in the liver. In some embodiments, the reticular vessel may be an intracranial vessel.

Blood flow parameters of the intracranial vessel are functional indicators that clinicians are eager to pay attention to. A determination of intracranial blood flow parameters plays an important role in prevention, diagnosis, treatment, and monitoring of reticular vessel diseases. However, it is difficult to model intracranial blood vessels, which have complex structures and numerous pathways, and it is extremely difficult to simulate hemodynamics of the intracranial blood vessels.

Computational Fluid Dynamics (CFD) technology is an interdisciplinary field between mathematics, fluid dynamics, and computer science, and is widely used in aerospace, automotive, turbine design, chemical processing industry, etc. Applying the CFD technology to a study of hemodynamics in a biomedical field may provide a unique perspective and role in clinical diagnosis and treatment practice. By modeling a target region and determining a flow distribution and a pressure distribution in the target region, corresponding hemodynamic indexes or parameters that are valuable to the clinic may be obtained.

Currently, there are some hemodynamic simulation methods for intracranial blood vessels in the related technology. But due to the complexity of the intracranial vessel structure, especially the presence of a Willis ring, these methods may only perform a simple simulation for localized vessels or unilateral vessels, and may not accurately simulate an entire reticular vessel including the Willis ring. The Willis ring, also known as a cerebral arterial ring, is a vessel ring structure located within the skull. The vessel ring structure includes three sets of arterial rings: anterior arterial ring, posterior arterial ring, and middle arterial ring, and the vessel ring structure connects major arteries that supply blood to the brain.

Some embodiments of the present disclosure provide a method and a system for determining a blood flow parameter of a reticular vessel. When a blood flow parameter of intracranial blood vessels is determined, a rapid simulation on the entire reticular vessel including the Willis ring may be performed, and blood flow parameters of the vessels may be accurately determined, thereby providing more comprehensive clinical assessment results.

In some embodiments, the method for determining the blood flow parameter of the reticular vessel described in the present disclosure not only performs a rapid simulation and accurate determination on the entire reticular vessel, but also allows for localized analysis of specific regions of the reticular vessel, thereby deeply analyzing the hemodynamic characteristics of specific regions or structures.

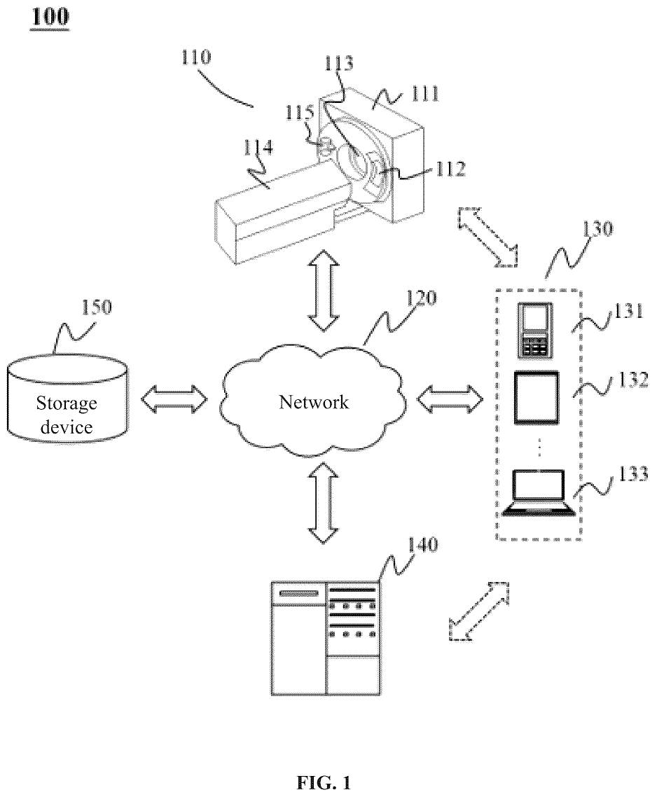

FIG. 1 is a schematic diagram illustrating an application scenario of a system for determining a blood flow parameter of a reticular vessel according to some embodiments of the present disclosure.

In some embodiments, a system 100 for determining a blood flow parameter of a reticular vessel (hereinafter referred to as system 100) may be widely used in many scenarios in the medical and scientific fields, including, but not limited to, diagnosis and treatment of vessel diseases, biomedical engineering research, drug discovery and development, and medical imaging diagnosis. Taking medical diagnosis as an example, in medical imaging, by determining a blood flow parameter (e.g., a blood flow rate, a blood flow velocity, a blood flow pressure, etc.), it may help doctors analyze and diagnose diseases (e.g., a heart disease, a cancer, or the like) more accurately.

As shown in FIG. 1, the system 100 may include a medical scanning device 110, a network 120, one or more terminals 130, a processor 140, and a storage device 150. Connections between components in the system 100 are variable. For example, the medical scanning device 110 may be connected to the processor 140 via the network 120. As another example, the medical scanning device 110 may be directly connected to the processor 140, as indicated by the dashed bi-directional arrow connecting the medical scanning device 110 to the processor 140. As another example, the storage device 150 may be connected to the processor 140 either directly or through the network 120. Merely by way of example, the terminal 130 may be connected directly to the processor 140 (as shown by the dashed arrow connecting the terminal 130 to the processor 140), or may be connected to the processor 140 via the network 120.

The medical scanning device 110 may be configured to perform a scan on an object to collect scanning data related to the object. The scanning data may be used to generate a medical image of the object. In some embodiments, the medical scanning device 110 may include a single modality scanner, e.g., a computed tomography (CT) device, a magnetic resonance imaging (MRI) device, a positron emission tomography (PET) device, an X-ray device (e.g., a digital subtraction angiography (DSA) device. a digital radiology (DR) device, etc.), an ultrasonic device, a single photon emission computed tomography (SPECT), or the like. In some embodiments, the medical scanning device 110 may also include a multimodal scanner, e.g., a computed tomography-positron emission tomography (CT-PET) device, a computed tomography-magnetic resonance imaging (CT-MRI) device, a positron emission tomography-magnetic resonance imaging (PET-MRI) device, a single photon emission computed tomography-magnetic resonance imaging (SPECT-MRI) device, a digital subtraction angiography-magnetic resonance imaging (DSA-MRI) device, or the like. The object may be biological or non-biological. Merely by way of example, the object may include a patient, an artificial object (e.g., an artificial mold), or the like. As another example, the object may include a specific part, an organ, and/or tissues of the patient.

In some embodiments, taking the CT or X-ray device as an example, the medical scanning device 110 may include a gantry 111, a detector 112, a detection region 113, a table 114, and a radioactive source 115. The gantry 111 may support the detector 112 and the radioactive source 115. The object may be located on the table 114 for scanning. The radioactive source 115 may emit a radiation (e.g., X-rays) to the object. The detector 112 may detect the radiation emitted by the radioactive source 115. In some embodiments, there may be a plurality of radioactive sources 115, which may scan the object at different scanning planes from different angles. In some embodiments, the detector 112 may include one or more detector units. Each of the detector units may include a scintillation detector (e.g., a cesium iodide detector), a gas detector, or the like. The detector units may include a single-line detector and/or a multi-line detector.

The network 120 may include any suitable network capable of facilitating an exchange of information and/or data in the system 100. In some embodiments, one or more components (e.g., the medical scanning device 110, the terminal 130, the processor 140, or the storage device 150) of the system 100 may exchange information and/or data with each other through the network 120. For example, the processor 140 may obtain vessel images from the medical scanning device 110 via the network 120. As another example, the processor 140 may obtain user instructions from the terminal 130 via the network 120.

The network 120 may be a public network (e.g., the Internet), a private network (e.g., a local region network (LAN), a wide region network (WAN), etc.), a wired network (e.g., an Ethernet network), a wireless network (e.g., an 802.11 network, a Wi-Fi network, etc.), a cellular network (e.g., a long-term evolution (LTE) network), a frame relay network, a virtual private network (VPN), a satellite network, a telephone network, a router, a hub, a switch, a server computer, or the like, or any combination thereof. In some embodiments, the network 120 may include one or more network access points. For example, the network 120 may include wired and/or wireless network access points, such as a base station and/or an Internet exchange point, through which one or more components of the system 100 may be connected to the network 120 to exchange data and/or information.

The terminal 130 may include a mobile device 131, a tablet computer 132, a laptop computer 133, etc., or any combination thereof. In some embodiments, the mobile device 131 may include a smart home device, a wearable device, a virtual reality device, an augmented reality device, etc., or any combination thereof. In some embodiments, the terminal 130 may be part of the processor 140.

The processor 140 may process data and/or information obtained from the medical scanning device 110, the terminal 130, and/or the storage device 150. For example, the processor 140 may obtain scanning data obtained by the medical scanning device 110 by scanning the object and utilize the scanning data for imaging to generate a medical image (e.g., a vessel image, etc.). As another example, the processor 140 may generate a reticular vessel model by performing, based on the vessel image, vessel segmentation; generate, by performing model partitioning based on the reticular vessel model, a plurality of vessel sub-models; determine the blood flow parameter of the reticular vessel model by performing, based on the plurality of vessel sub-models, coupling processing.

In some embodiments, the processor 140 may be a single server or a group of servers. The group of servers may be centralized or distributed. In some embodiments, the processor 140 may be local or remote. For example, the processor 140 may access information and/or data stored in the medical scanning device 110, the terminal 130, and/or the storage device 150 via the network 120. As another example, the processor 140 may be directly connected to the medical scanning device 110, the terminal 130, and/or the storage device 150 to access the stored information and/or data. In some embodiments, the processor 140 may be implemented on a cloud platform.

The storage device 150 may store data, instructions, and/or any other information. In some embodiments, the storage device 150 may store data obtained from the medical scanning device 110, the terminal 130, and/or the processor 140. For example, the storage device 150 may store medical image data (e.g., raw scanning data, vessel images, etc.) and/or positioning information data obtained from the medical scanning device 110. As another example, the storage device 150 may store at least one of images or scanning protocols input from the terminal 130.

In some embodiments, the storage device 150 may store data and/or instructions that the processor 140 performs or are used to perform the exemplary methods described in the present disclosure. In some embodiments, the storage device 150 includes a mass storage device, a removable storage device, a volatile read-write memory, a read-only memory (ROM), or the like, or any combination thereof. Exemplary mass storage devices may include a hard disk, an optical disk, a solid-state drive, etc. In some embodiments, the storage device 150 may be implemented on a cloud platform.

In some embodiments, the storage device 150 may be connected to the network 120 to communicate with one or more other components (e.g., the processor 140, and the terminal 130) in the system 100. One or more components of the system 100 may access data or instructions stored in the storage device 150 via the network 120. In some embodiments, the storage device 150 may be directly connected to or in communication with one or more other components (e.g., the processor 140, and the terminal 130) of the system 100. In some embodiments, the storage device 150 may be a part of the processor 140.

The description of the system 100 is intended to be illustrative and is not intended to limit the scope of the present disclosure. Many alternatives, modifications, and variations will be apparent to one of ordinary skill in the art. It will be appreciated that to ordinary skill in the art, under the teachings of the present disclosure, it is possible to arbitrarily combine the modules or form a subsystem connected with other modules without deviating from the principle.

Some embodiments of the present disclosure provide a method for determining a blood flow parameter of a reticular vessel, implemented on a device including at least one processor and at least one storage device. The method may include: obtaining vessel data of an object; generating a reticular vessel model by performing, based on the vessel data, vessel segmentation; generating, by performing model partitioning based on the reticular vessel model, a plurality of vessel sub-models; and determining the blood flow parameter of the reticular vessel model by performing, based on the plurality of vessel sub-models, coupling processing.

The vessel data refers to information related to a blood vessel, which is used to reflect structural or morphological characteristics of the vessel. In some embodiments, the vessel data may include anatomical structure data, e.g., a geometric shape of the vessel, a branch point, a curvature, and a distribution pattern and a positional relationship of the vessel within a specific tissue or organ. In some embodiments, the vessel data may include at least vessel image.

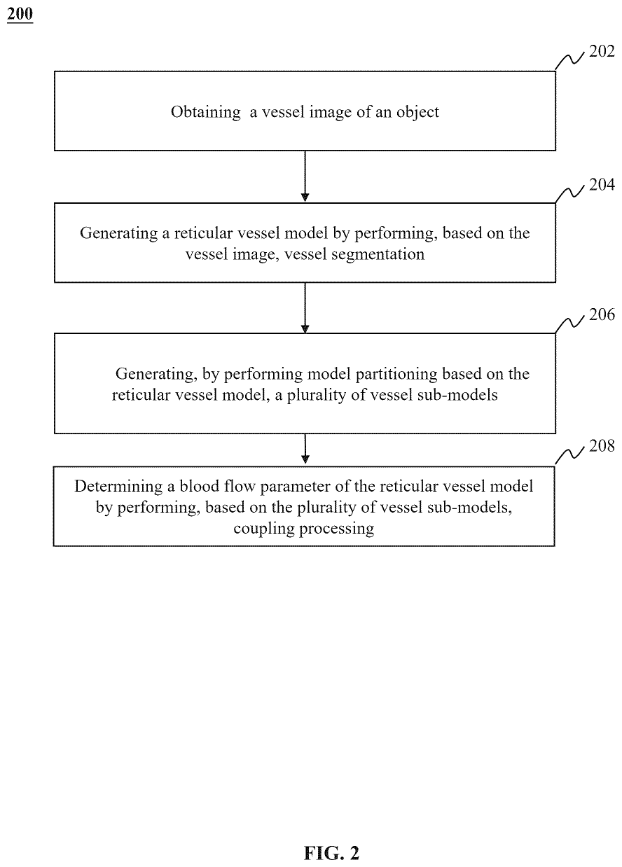

FIG. 2 is an exemplary flowchart illustrating an exemplary process for determining a blood flow parameter of a reticular vessel according to some embodiments of the present disclosure. In some embodiments, process 200 may be performed by the system for determining a blood flow parameter of a reticular vessel or a processor (e.g., the processor 140). As shown in FIG. 2, the process 200 includes the following operations.

In 202, a vessel image of an object may be obtained. In some embodiments, operation 202 may be performed by a data acquisition module 610.

The object includes at least a portion of a biological (e.g., patients, animals, etc.) and/or non-biological object (e.g., a phantom) in a scanning process. For example, the object may be a region including the head.

The vessel image refers to a medical image that includes a vessel structure of the object. The vessel image may be a vessel image of one or more parts of the body of the object, for example, the vessel image is a vessel image of the brain of the object (also referred to as a cerebrovascular image). In some embodiments, the vessel image may be a two-dimensional image or a three-dimensional image.

In some embodiments, the vessel image includes a reticular vessel.

In some embodiments, the vessel image may include an original image and/or a post-processing image. Scanning data may be obtained by scanning the object using the medical scanning device 110, and then image reconstruction may be performed on the scanning data to obtain an original image. For example, scanning data may be obtained by scanning the brain of the object with a CT device, and then image reconstruction may be performed on the scanning data to obtain a cerebrovascular image. The cerebrovascular image may be used for detecting and diagnosing cerebrovascular diseases such as stroke, aneurysm, cerebrovascular malformation, or the like. In some embodiments, post-processing may be performed on at least one medical image to obtain a post-processing image. The post-processing may include, but is not limited to, maximum intensity projection (MIP), multi-plane reconstruction (MPR), curved planar reformation (CPR), three-dimensional rendering, etc.

In some embodiments, at least one vessel image may be obtained. For example, vessel images from different angles and/or planes may be obtained. For example, the vessel images may include cross-sectional, sagittal, and coronal views of a vessel structure of the object. In some embodiments, vessel images determined from different angles or different planes may be segmented, and a reticular vessel model may be obtained based on the segmentation result of the vessel images.

In some embodiments, the processor may obtain a vessel image of the object by accessing the storage device (e.g., storage device 150) or a database.

In 204, a reticular vessel model may be generated by performing, based on the vessel image, vessel segmentation. In some embodiments, operation 204 may be performed by a vessel model generation module 620.

The reticular vessel model may be a model that reflects a topology of the reticular vessel. The topology may include nodes and edges. The node represents an entry point, an exit point, a branch point, or a confluence point of the blood flow in the reticular vessel, and the edge represents a blood vessel connecting two nodes. The branch point is a location in the reticular vessel where a single vessel or pathway divides into two or more branches. The confluence point is a location in the reticular vessel where two or more vessels or pathways merge into a single vessel or pathway. In some embodiments, branch points and confluence points are also referred to as connection points.

The reticular vessel model is a three-dimensional model constructed to demonstrate geometry of a reticular vessel. For example, the reticular vessel model may be a three-dimensional model constructed in a three-dimensional coordinate system having contours of blood vessels of the object. In the three-dimensional model, a shape of the blood vessel is similar to a reticular shape, and therefore the blood vessel is referred to as the reticular vessel model.

The vessel segmentation is a process for identifying or extracting blood vessels from the vessel image to distinguish the blood vessels from other tissues or background. By processing and analyzing medical images, the vessel segmentation is able to extract the blood vessels, and label the extracted blood vessels in the vessel image or generate an image only presenting the extracted blood vessels.

In some embodiments, the processor may perform the vessel segmentation on the vessel image using a blood vessel segmentation algorithm. The blood vessel segmentation algorithm may include a thresholding-based manner, an edge detection algorithm-based manner, a region growing-based manner, a deep learning-based manner, etc.

In some embodiments, the processor may generate the reticular vessel model based on a vessel segmentation result by reconstructing using surface reconstruction, voxel reconstruction, or curved planar reformation. Specific reconstruction algorithms include a deep learning algorithm, marching cubes, marching tetrahedra, level set, etc., and the embodiment does not limit the specific reconstruction algorithms used as long as the corresponding functions are achieved.

In 206, a plurality of vessel sub-models may be generated by performing model partitioning based on the reticular vessel model. In some embodiments, operation 206 may be performed by a model partitioning module 630.

The plurality of vessel sub-models refer to a plurality of small models that are independent of each other in structure but to associated with each other by connections, and are obtained by segmenting the reticular vessel model. As used herein, “independent of each other in structure” refers to the absence of overlapping parts between the structures of the vessel sub-models, indicating structural independence, and “associated with each other by connections” refers to that there are connections (e.g., relative pre-order and relative post-order relationships between two vessel sub-models) among the vessel sub-models. For example, a vessel sub-model may be a part of the reticular vessel model. By segmenting the reticular vessel model into a plurality of sub-models, the properties and behaviors of the reticular vessel model may be understood and investigated in greater depth, leading to better determination of the blood flow parameter.

The partitioning is a process for segmenting the reticular vessel model and determining connections of the vessel sub-models after the segmentation.

The segmentation refers to segmenting the reticular vessel model to obtain the plurality of vessel sub-models. The connections refer to ways in which the vessel sub-models are connected to each other. More details about the connections of the vessel sub-models can be found in the description below.

In some embodiments, the segmentation manner of the reticular vessel model may be determined based on the vessel data within the reticular vessel model using a graph partitioning algorithm, such as spectral clustering, a minimum cut algorithm, or K-means clustering. Through the graph partitioning algorithm, the reticular vessel model may be effectively segmented into the plurality of vessel sub-models according to the geometric and/or functional characteristics of the vessels.

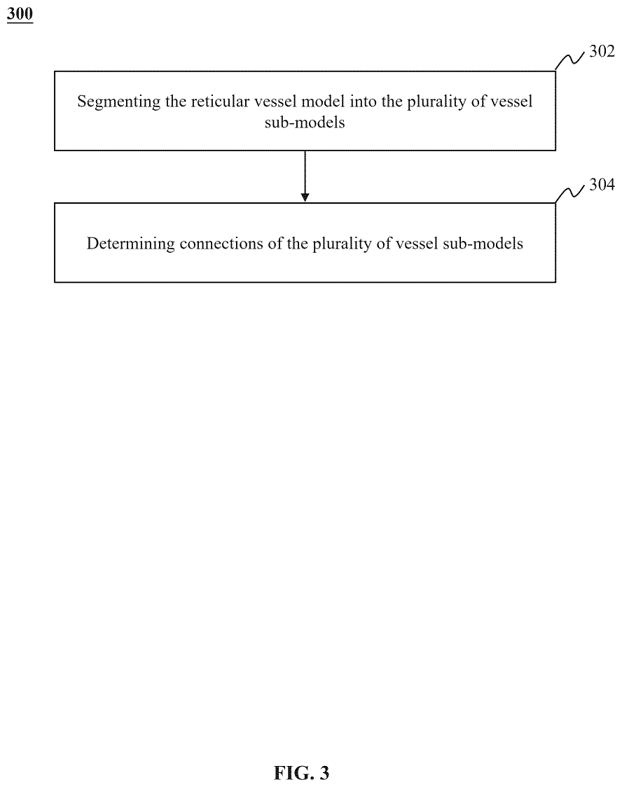

In some embodiments, hemodynamic simulations may be used to determine the blood flow direction within and between the vessel sub-models to determine the connection between the vessel sub-models. The operation of determining the connection between the vessel sub-models may include: setting an initial boundary parameter, simulating a blood flow path, and determining a vessel connection direction. For example, setting the initial boundary parameter may include setting initial boundary parameters such as an inlet blood flow velocity, a blood flow rate, and a blood viscosity for each of the vessel sub-models using computational fluid dynamics or other numerical simulation algorithms. More details regarding the process for partitioning the reticular vessel model may be found in other contents of the present disclosure (e.g., description in connection with FIG. 3).

In 208, the blood flow parameter of the reticular vessel model may be determined by performing, based on the plurality of vessel sub-models, coupling processing. In some embodiments, operation 208 may be performed by a coupling processing module 640.

The coupling processing refers to a process for coupling the vessel sub-models based on the connection of the vessel sub-models and determining the blood flow parameter of the coupled vessel sub-models. The coupling may include determining relative pre-order and relative post-order relationships (also referred to as upstream and downstream relationships) of two vessel sub-models.

In some embodiments, coupling processing refers to the process of reconnecting or linking the vessel sub-models that were previously segmented during model partitioning. For example, after segmenting a reticular vessel into sub-models, coupling could involve rejoining these sub-models at their cut-off surfaces to ensure they accurately represent continuous blood flow through the entire vessel network. This process ensures that the blood flow parameter is correctly determined by taking into account the interactions at the points where the sub-models are reconnected.

The blood flow parameter may include a series of physical quantities and indicators of a state and property of blood when the blood flows through blood vessels. The blood flow parameter may include one or more of a blood flow direction, a blood flow velocity, a blood flow pressure, a blood flow rate, a blood flow resistance, a shear force, a vessel diameter, a blood viscosity, or a vessel elasticity. In some embodiments, the blood flow parameter may be used to assess and diagnose vessel disease, guide clinical treatments and surgical planning, or the like.

Blood flow parameter determination refers to a process for determining the blood flow velocity, the blood flow pressure, the blood flow rate, the blood flow resistance, etc., of blood in a blood vessel in a certain way, e.g., by way of computational fluid dynamics or deep learning models.

In some embodiments, the coupling processing includes a process for determining a blood flow parameter of a relative pre-order sub-model based on physiological data of an object, determining, based on a connection and the blood flow parameter of the relative pre-order sub-model, a boundary parameter of a relative post-order sub-model corresponding to the relative pre-order sub-model, and determining, based on the boundary parameter, a blood flow parameter of the relative post-order sub-model.

In some embodiments, the boundary parameter is included as part of the blood flow parameters of a sub-model. For example, the boundary parameter includes one or more of a blood flow pressure, a blood flow velocity, a blood flow rate and a blood flow resistance of the relative post-order sub-model.

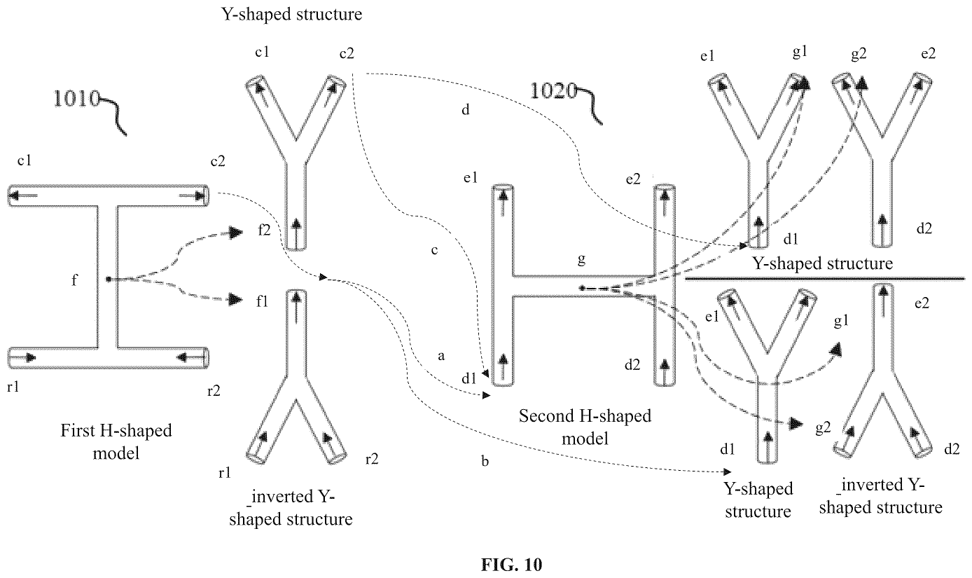

In some embodiments, the boundary parameter (e.g., the blood flow parameter of the inlet) of one vessel sub-model (assume as the first vessel sub-model) may be determined based on the blood flow parameter of the outlet of another one vessel sub-model (assume as the second vessel sub-model). In some embodiments, if an outlet of the first vessel sub-model and an inlet of a the second vessel sub-model correspond to the same location of the reticular vessel model that is neither a confluence point nor a branch point, and there is no collision flow in the first vessel sub-model and the second vessel sub-model, values of the blood flow parameters of the outlet of the first vessel sub-model and the inlet of the second vessel sub-model at the same location are the same. For example, referring to FIG. 10, one type is to segment the first H-shaped model into one basic sub-model with Y-shaped structure and one basic sub-model with inverted Y-shaped structure. In this type, the blood flows from r₁ to f₁ and from f₂ to c₂ (f₁ and f₂ may actually be considered as the same point and correspond to point f). And therefore, the value of the blood flow parameter at f₁ and the value of the blood flow parameter at f₂ are the same.

In some embodiments, if an outlet of a relative pre-order sub-model and inlets of relative post-order sub-models correspond to the same location of the reticular vessel model that is a branch point, values of the blood flow parameter of the outlet of the relative pre-order sub-model is the sum of values of the blood flow parameters of the inlets of the relative post-order sub-models. The value of blood flow parameter corresponding to each inlet of the relative post-order sub-models may be determined based on the diameter of the vessel corresponding to the inlet. For example, the larger the vessel diameter, the greater the blood flow parameter value of the inlet. As another example, a ratio of the vessel diameters of the inlets of the relative post-order sub-models may be equal to a ratio of the blood flow parameters of the inlets of the relative post-order sub-models. For instance, the blood flow volume of the outlet of the relative pre-order sub-model may be 50 ml/min. A ratio of the vessel diameters of the inlets of two relative post-order sub-models A and B may be 2:3. Accordingly, the blood flow volumes of the inlets of the two relative post-order sub-models A and B are 20 ml/min and 30 ml/min, respectively. If outlets of relative pre-order sub-models and an inlet of a relative post-order sub-model correspond to the same location of the reticular vessel model that is a confluence point, values of the blood flow parameter of the inlet of the relative post-order sub-model is the sum of values of the blood flow parameters of the outlets of the relative pre-order sub-models.

In some embodiments, the first relative pre-order sub-model refers to a sub-model that has no relative pre-order sub-model. For example, the sub-model including at least one inlet of the reticular vessel model may be regarded as the first relative pre-order sub-model. The boundary parameter of the first relative pre-order sub-model includes one or more blood flow parameters of at least one inlet of the reticular vessel model. The one or more blood flow parameters of at least one inlet of the reticular vessel model may be determined based on physiological data of the object. For example, the one or more blood flow parameters of at least one inlet of the reticular vessel model may be obtained through various medical measurement techniques. Merely by way of example, a blood flow pressure of the inlet of the reticular vessel model may be determined by a blood pressure meter, and a blood flow velocity of the inlet of the reticular vessel model may be determined by Doppler ultrasound or MRI, et al. Furthermore, based on the blood flow velocity and the cross-sectional area of the vessel, the blood flow rate may be determined. In some embodiments, the blood flow velocity, the blood flow rate, or the blood flow resistance of the outlet of the reticular vessel model may be determined based on the blood flow velocity and the blood flow rate of the inlet of the reticular vessel model and a size of the outlet of each of the plurality of vessel sub-models.

In some embodiments, for a sub-model, one or more boundary parameters of each outlet of the sub-model may be determined based on Murray's law and a size (e.g., a blood diameter) of the outlet. For example, a blood flow rate of each outlet of the sub-model may be determined based on Equations (1) and (2):

$${Q}_{{out}_{i}} = a \cdot {d}_{{out}_{i}}^{k} \qquad ( 1 )$$

$${Q}_{i n} = {\sum }_{i = 1}^{n} {Q}_{{out}_{i}} \qquad ( 2 )$$

wherein Qᵢₙ refers to a blood flow rate of an inlet of the sub-model; Qₒᵤₜ_{ᵢ} refers to a blood flow rate of outlet i of the sub-model, and n refers to a count of outlets of the sub-model; dₒᵤₜ_{ᵢ} refers to a blood diameter of outlet i of the sub-model; k refers to a Murray's exponent, such as 3.0, 2.7, 2.5, etc.; and a refers to a constant coefficient.

In some embodiments, for a sub-model, one or more blood flow parameters of the sub-model may be determined based on one or more boundary parameters of the sub-model. A distal vessel resistance at each outlet of the sub-model may be determined based on the one or more blood flow parameters of the sub-model. For example, a distal vessel resistance at each outlet of the sub-model may be determined based on Ohm's law, and a blood pressure and a blood flow rate of the outlet of the sub-model. Merely by way of example, the distal vessel resistance at each outlet of the sub-model may be determined based on Equation (3):

$${R}_{{out}_{i}} = \frac{{P}_{o u {t}_{i}}}{{Q}_{o u {t}_{i}}} \qquad ( 3 )$$

wherein Rₒᵤₜ_{ᵢ} refers to the distal vessel resistance at outlet i of the sub-model; and Pₒᵤₜ_{ᵢ} refers to the blood pressure at outlet i of the sub-model.

In some embodiments, the blood flow parameter of the relative post-order sub-model includes blood flow parameters at an inlet and an outlet of the relative post-order sub-model and a result of an overall 3D model. The result of the overall 3D model refers to a flow field distribution of a meshed vessel three-dimensional model corresponding to the relative post-order sub-model. The vessel sub-model that the blood flowing out is a relative pre-order sub-model, and the vessel sub-model that the blood flowing into is a relative post-order sub-model.

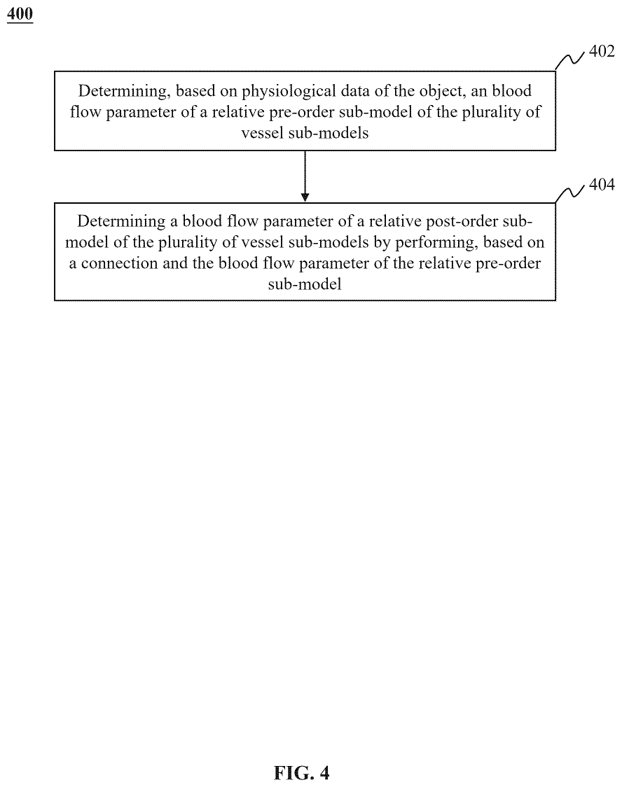

In some embodiments, the blood flow parameter determination may be performed in a variety of ways. For example, the processor may perform the blood flow parameter determination through Computational Fluid Dynamics (CFD) or a machine learning model. More details regarding the determination of the blood flow parameter may be found in other contents of the present disclosure (e.g., description in connection with FIG. 4), which will not be repeated here.

In some embodiments, the processor may perform an application analysis based on the blood flow parameter of the reticular vessel model. For example, the processor may perform a clinical diagnostic analysis.

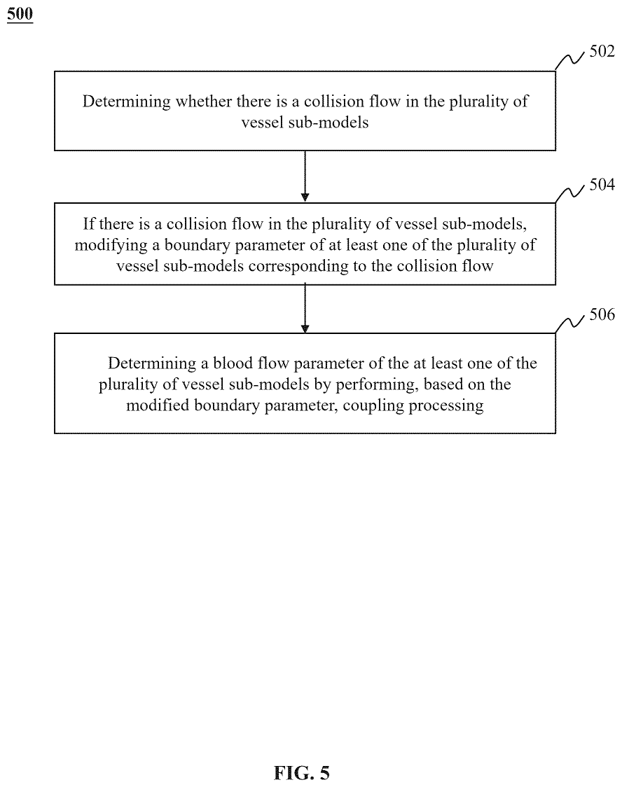

In some embodiments, the processor may determine a blood flow parameter of a vessel sub-model in a specific region using different determination manners. The specific region may include complex and critical regions such as a Willis ring region, a lesion of the object, a region within a preset range of the lesion, or a region with a collision flow. More details regarding the region within a preset range of the lesion may be found in other contents of the present disclosure (e.g., description in connection with FIG. 3). More details regarding the collision flow may be found in other contents of the present disclosure (e.g., description in connection with FIG. 5). For example, for the specific region, the processor may use at least two different manners to determine the blood flow parameter. Exemplarily, the processor may utilize CFD and a machine learning model to determine the blood flow parameter. Using different manners to determine the blood flow parameter may result in different results of blood flow parameter, and the processor may take an average of the different results of blood flow parameter as a final blood flow parameter.

By determining the blood flow parameter of the vessel sub-model in a specific region using different determination manners, an accuracy of blood flow parameter determination in critical regions or sections of the vessels may be improved, which facilitates accurate diagnosis by doctors of the critical region, thereby ultimately benefiting the effectiveness of subsequent treatments.

In some embodiments, the processor may determine the blood flow parameter of the vessel sub-model in the specific region using a specific machine learning model. The machine learning model may be a Neural Network (NN), a Convolutional Neural Network (CNN), or other models.

In some embodiments, an input of the machine learning model may include physiological data of the object and the vessel sub-model in the specific region, and an output of the machine learning model may be the blood flow parameter of the vessel sub-model in the specific region.

In some embodiments, the specific machine learning model may be trained based on labeled training samples. The training samples may include sample physiological data of a sample object and a sample vessel sub-model in a sample region. The label may be an actual blood flow parameter of the sample vessel sub-model. In some embodiments, the training samples and the labels may be obtained from historical clinical treatment records.

In some embodiments, the processor may train the machine learning model using manners such as gradient descent. The processor may input a plurality of labeled training samples into an initial machine learning model, construct a loss function based on the labels and output results of the initial machine learning model, and iteratively update parameters of the initial machine learning model based on the loss function. The training may be completed when the loss function of the initial machine learning model meets a preset condition, and then a trained machine learning model may be obtained. The preset condition may include the loss function converging, a count of iterations reaching a threshold, etc.

Using the specific machine learning model to predict the blood flow parameter of the vessel sub-model in the specific region, while employing conventional manners (e.g., simpler manners like CFD) for other regions, blood flow parameter determination may be more targeted, thereby improving the accuracy of blood flow parameter prediction.

In some embodiments of the present disclosure, a three-dimensional reticular vessel model is constructed for the blood flow parameter determination based on a medical image of the object. The blood flow parameter determination is more accurate due to the partition process and the coupling process performed on the reticular vessel model. For example, compared to the existing manner of determining the blood flow parameter directly using the entire reticular vessel model without segmenting the reticular vessel model, determining the blood flow parameters of the vessel sub-models is more focused, which can improve the accuracy of the blood flow parameter determination result. At the same time, the blood flow parameter determination process is performed on the vessel sub-models obtained by segmenting the reticular vessel model, and thus has strong universality. It can provide non-invasive hemodynamic assessment results for intracranial vessels, and stable and effective determination of blood flow parameters for the reticular vessel models including a Willis ring, providing comprehensive hemodynamic assessment data.

FIG. 3 is an exemplary flowchart illustrating an exemplary process for partitioning according to some embodiments of the present disclosure. In some embodiments, process 300 may be performed by the system for determining a blood flow parameter of a reticular vessel or a processor (e.g., the processor 140). As shown in FIG. 3, the process 300 includes the following operations.

In 302, a reticular vessel model may be segmented into a plurality of vessel sub-models.

The processor may segment a reticular vessel model using a plurality of manners. In some embodiments, the processor may randomly segment the reticular vessel model.

In some embodiments, the processor may generate a plurality of candidate segmentation manners. For each of the plurality of candidate segmentation manners, the processor may predict, through a prediction model, an accuracy of blood flow parameter determination performed using the candidate segmentation manner. The processor may segment the reticular vessel model into the plurality of vessel sub-models based on the candidate segmentation manner corresponding to a highest accuracy.

The candidate segmentation manner refers to a manner configured to perform a segmentation on the reticular vessel model. For example, the candidate segmentation manners may include a segmentation region, a count of vessel sub-models obtained by the segmentation, or the like. For example, a candidate segmentation manner may be to segment the reticular vessel model into a plurality of vessel sub-models with the same volume. In some embodiments, a candidate segmentation manner may be preset. In some embodiments, a candidate segmentation manner may be a segmentation manner used in historical clinical treatments.

The accuracy of the blood flow parameter determination may reflect an accuracy of the blood flow parameter determination of the reticular vessel model performed by the candidate segmentation manner, which in turn may reflect a rationality of the candidate segmentation manner. The higher the accuracy of the blood flow parameter determination, the more reasonable the corresponding candidate segmentation manner.

The prediction model is a model used to predict the accuracy of the blood flow parameter determination in the reticular vessel model. In some embodiments, the prediction model may be a machine learning model, such as a Neural Network (NN) model, a Convolutional Neural Network (CNN) model, or the like.

In some embodiments, an input of the prediction model may include a candidate segmentation manner, a vessel image of an object, and physiological data of the object; and an output of the prediction model may include an accuracy of blood flow parameter determination of the reticular vessel model performed using the candidate segmentation manner.

More details regarding the object and the vessel image of the object may be found in other contents of the present disclosure (e.g., description in connection with FIG. 2). More details regarding the physiological data of the object may be found in other contents of the present disclosure (e.g., description in connection with FIG. 4).

In some embodiments, the processor may obtain the prediction model based on a large number of labeled training samples. The training samples may include a sample vessel image of a sample object, sample physiological data of the sample object, and an actual segmentation manner of the sample reticular vessel model. In some embodiments, the training samples may be obtained based on historical clinical treatment records. The label may be a similarity between an actual blood flow parameter and a predicted blood flow parameter. The actual blood flow parameter refers to a blood flow parameter of the sample reticular vessel model corresponding to the sample vessel image; and the predicted blood flow parameter refers to a blood flow parameter that is predicted by a determination manner of the blood flow parameter provided in the present disclosure through the actual segmentation manner corresponding to the sample object. In some embodiments, the actual blood flow parameter may be obtained through existing technologies, e.g., angiography, color Doppler ultrasound, etc. More details regarding the determination manner of the blood flow parameter may be found in other contents of the present disclosure (e.g., description in connection with FIG. 4).

In some embodiments, the processor may train the prediction model using manners such as gradient descent. The processor may input a plurality of labeled training samples into an initial prediction model, construct a loss function based on the label and output results of the initial prediction model, and iteratively update parameters of the initial prediction model based on the loss function. The training is complete when the loss function of the initial prediction model meets a preset condition, and a trained prediction model may be obtained. The preset condition may include the loss function converging, the count of iterations reaching a threshold, etc.

Using the prediction model to predict the accuracy of the blood flow parameter determination of the reticular vessel model corresponding to each of the plurality of candidate segmentation manners, the effect of the plurality of candidate segmentation manners may be evaluated, which avoids the need to actually perform each of the plurality of candidate segmentation manners to determine the better segmentation manner, thereby saving processing resources.

In some embodiments, the processor may determine, based on the accuracy obtained by the prediction model, the candidate segmentation manner corresponding to the highest accuracy as the segmentation manner ultimately used to perform the segmentation on the reticular vessel model.

In some embodiments, the prediction model may be used to predict a computing resource consumed by the candidate segmentation manner. The computing resource refers to a resource required for performing a task (e.g., generating, by performing model partitioning based on the reticular vessel model, a plurality of vessel sub-models and/or determining the blood flow parameter of the reticular vessel model by performing coupling processing on the plurality of the vessel sub-models). For example, the computing resource may include CPU resources, memory resources, network resources, or the like. In some embodiments, the computing resource may be reflected by a processing duration.

The processing duration refers to a total time consumed to generate, by performing model partitioning on the reticular vessel model based on the candidate segmentation manner, a plurality of vessel sub-models, and/or determine the blood flow parameter of the reticular vessel model by performing coupling processing on the plurality of the vessel sub-models obtained based on the candidate segmentation manner. The longer the processing duration, the more the computing resources may be consumed.

In some embodiments, when the prediction model is used to predict the computing resource consumed by the candidate segmentation manner, an input of the prediction model may include the candidate segmentation manner and the reticular vessel model, and an output of the prediction model may include a processing duration consumed by the candidate segmentation manner.

In some embodiments, the processor may obtain the prediction model based on a large number of labeled training samples to predict the computing resources consumed by the candidate segmentation manner. The training samples may include an actual segmentation manner and a sample reticular vessel model. The labels may be an actual processing duration consumed to determine the blood flow parameter of the sample reticular vessel model. The training process of the prediction model in this embodiment is similar to the training process of the prediction model described previously, which is not described herein.

By determining the processing duration by the prediction model, manual resources may be saved and the efficiency of processing duration determination may be improved. Reflecting the consumption of the computing resources through the length of the processing duration simplifies the complex process of determining the computing resources and facilitates an intuitive result of the computing resources.

In some embodiments, the processor may determine the segmentation manner ultimately used to perform the segmentation on the reticular vessel model based on the accuracy obtained from the prediction model and the processing duration. For example, based on an accuracy threshold, the processor may select a candidate segmentation manner with a shortest processing duration as the segmentation manner that is ultimately used to perform the segmentation on the reticular vessel model among the candidate segmentation manners corresponding to the accuracy that are above the accuracy threshold. The accuracy threshold may be obtained by a person skilled in the art in a preset manner. As another example, the processor may determine the segmentation manner ultimately used to perform the segmentation on the reticular vessel model through a weighting process based on weighted results. Weights for the accuracy and the processing duration may be determined based on actual needs. For example, the processor may assign a first weight to the accuracy, assign a second weight to the processing duration. The higher the accuracy, the greater the first weight. The longer the processing duration, the smaller the second weight. For each candidate segmentation manner, the processor may determine a weighted result based on the accuracy, the processing duration, the first weight, and the second weight, and determine the candidate segmentation manner corresponding to a maximum weighted result as the segmentation manner that is ultimately used to segment the reticular vessel model.

Through determining the segmentation manner that is ultimately used to segment the reticular vessel model based on the plurality of different candidate segmentation manners, a more rational segmentation of the reticular vessel model may be realized, which can minimize the processing duration and conserve resources while improving the accuracy of blood flow parameter determination.

In some embodiments, the plurality of vessel sub-models may include a plurality of composite sub-models and/or a plurality of basic sub-models.

In some embodiments, the processor may determine a segmentation refining degree of the reticular vessel model, and segment the reticular vessel model based on the segmentation refining degree. The segmentation refining degree refers to a refining degree at which the reticular vessel model is segmented. For example, blood vessels in regions with a relatively simple organ or tissue structure may be subjected to a relatively rough segmentation; while blood vessels in regions with a relatively complex organ or tissue structure may be subjected to a relatively detailed segmentation. For example, vessels of a certain region are segmented into a plurality of vessel sub-models. If each of the vessel sub-models includes more basic sub-models, it means that each of the vessel sub-models has a larger area and a count of the plurality of vessel sub-models is less, i.e., the more basic sub-models included in each of the vessel sub-models, the rougher the segmentation is. If each of the vessel sub-models includes fewer basic sub-models, it means that each of the vessel sub-models has a smaller area and a count of the plurality of vessel sub-models is greater, i.e., the fewer basic sub-models included in each of the vessel sub-models, the more detailed the segmentation is.

In some embodiments, the segmentation refining degree may be determined by a person skilled in the art based on experience.

In some embodiments, the processor may determine the segmentation refining degree based on a type of a region. For example, for a lesion of the object and blood vessels in a region within a preset range of the lesion, the processor may perform a relatively detailed segmentation, and perform a relatively rough segmentation on other regions. The preset range may be determined based on lesion information (e.g., a lesion type, a lesion shape, a lesion location, and a lesion size). For example, the larger the lesion or the deeper the lesion location, the larger the preset range.

By performing a relatively detailed segmentation for the lesion of the object and blood vessels in the region within the preset range of the lesion, and performing a relatively rough segmentation on other regions, on the one hand, it can help the doctors to observe the situation of the lesion clearly and facilitate the clinical diagnosis, and on the other hand, the computing resource may be saved while ensuring the accuracy.

In some embodiments, for each of the plurality of candidate segmentation manners, blood vessels in different regions may have different segmentation refining degrees. For example, the candidate segmentation manner may be to segment the diseased region a plurality of times and segment a region surrounding the diseased region once.

Using different segmentation refining degrees to segment different regions of blood vessels can save resources while ensuring the accuracy.

By segmenting the reticular vessel model based on the segmentation refining degree, the key regions may be segmented in detail, while the regions with uncomplicated structure or less attention may be roughly segmented, which can ensure the accuracy while saving resources.

In some embodiments, the processor may segment the reticular vessel model to obtain the plurality of vessel sub-models, and determine connections of the vessel sub-models The connections are related to at least one blood flow direction of the plurality of vessel sub-models and at least one correspondence of vessel branches of the different vessel sub-models. The correspondence of vessel branches of the different vessel sub-models refers to the structural or functional matching or association between the vessel branches in different sub-models. In some embodiments, the correspondence of vessel branches of the different vessel sub-models may manifest as the branches where blood flows out in one vessel sub-model corresponding to the branches where blood flows in within another vessel sub-model. For example, referring to FIG. 10, one type is to segment the second H-shaped model into two basic sub-models with Y-shaped structure. In this type, the blood flows from d₁ to g₁ and from d₂ to g₂ (g₁ and g₂ may actually be considered as the same point and correspond to point g).

In some embodiments, when partitioning the reticular vessel model into the plurality of vessel sub-models, the blood flow direction of each vessel sub-model and the connections between the plurality of vessel sub-models may be determined. During coupling of the partitioned vessel sub-models, if two different vessel sub-models are connected at a certain endpoint, the blood flow direction at that endpoint may be the same or opposite. If the blood flow direction at that endpoint is opposite, this indicates the presence of a collision flow between the two vessel sub-models.

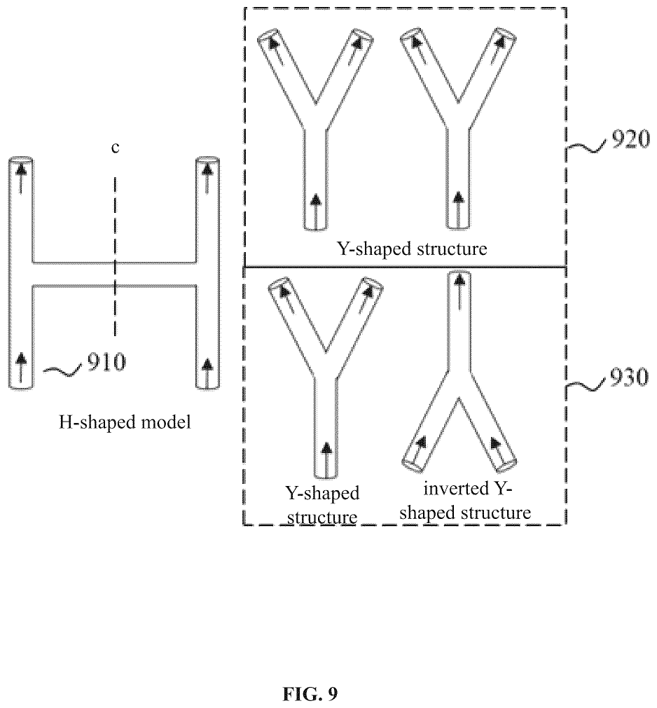

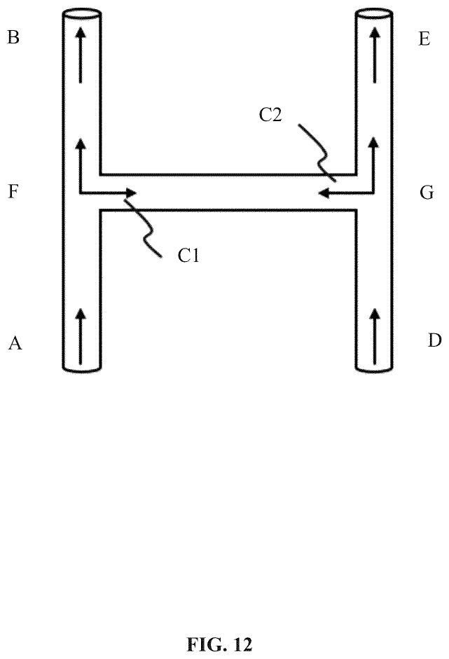

For example, if two vessel sub-models are coupled into an H-shaped model, and the blood flow in this H-shaped model enters from the lower ports (e.g., nodes A and D shown in FIG. 12) while exiting through the upper ports (e.g., nodes B and E shown in FIG. 12), or enters from the upper ports while exiting through the lower ports, this situation may lead to a collision flow between the two vessel sub-models. Merely by way of example, as shown in FIG. 12, an H-shaped model is partitioned into a first sub-model and a second sub-model at a partition point on vessel FG. The first sub-model (the sub-model on the left) includes 4 nodes (denoted as A, F, B, and C₁) and 3 edges (denoted as AF, FB, and FC₁), and the blood flow direction is determined, when partitioning, as from A to F, from F to B, and from F to C₁. The second sub-model (the sub-model on the right) includes 4 nodes (denoted as D, G, C₂, and E) and 3 edges (denoted as DG, GC₂, and GE), C₁ and C₂ correspond to the partition point, and the blood flow direction is determined, when partitioning, as from D to G, from G to C₂, and from G to E. During coupling of the first and second sub-models, it is clear that the blood flow direction at the partition point is opposite, which indicates the presence of a collision flow between the first and second sub-models. Details regarding the H-shaped model can be found elsewhere in the present disclosure (e.g., FIGS. 9, 10, and 12). Details regarding the collision flow can be found elsewhere in the present disclosure (e.g., FIGS. 5, 10, and 12).

In some embodiments, if there is no collision flow between two vessel sub-models, there is no need to adjust the boundary parameters during the coupling processing. If there is a collision flow between two vessel sub-models, the boundary parameters of one of the two vessel sub-models corresponding to the collision flow need to be modified during the coupling processing. Based on the modified boundary parameters, the blood flow parameters of one of the two sub-models are redetermined. Details regarding modifying the boundary parameters of the vessel sub-model corresponding to the collision flow can be found elsewhere in the present disclosure (e.g., FIGS. 5 and 12).

In some embodiments, the processor may segment the reticular vessel model into the plurality of vessel sub-models with a predetermined model segmentation manner (e.g., the candidate segmentation manner). The predetermined model segmentation manner may be a segmentation manner based on a hierarchical structure of the reticular vessel. For example, the predetermined model segmentation manner may include segmenting the reticular vessel into different levels of blood vessels such as a trunk blood vessel, a branch blood vessel, a terminal blood vessel, or the like, or segmenting the reticular vessel into blood vessels with different shapes such as a H-shaped, a Y-shaped, an inverted Y-shaped, etc.

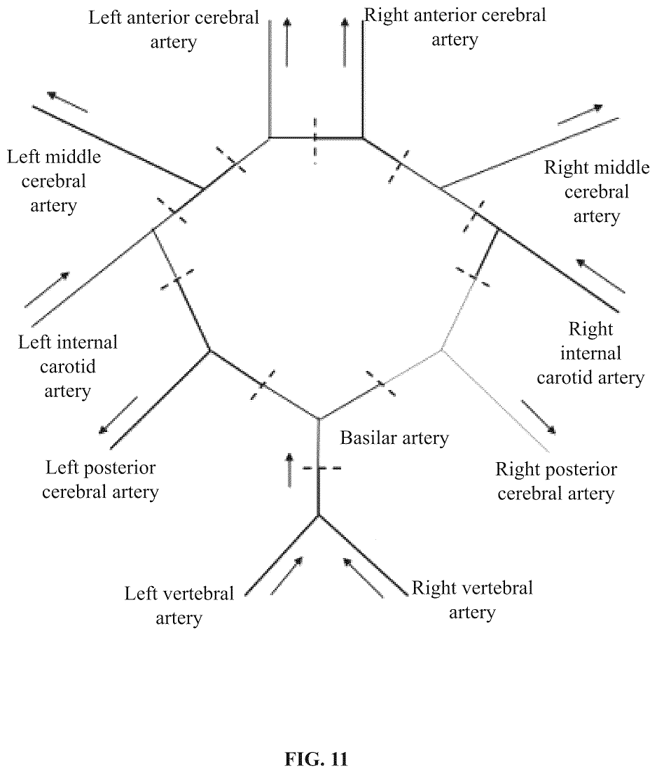

The basic sub-model refers to a most basic model of the model segmentation. In some embodiments, the basic sub-model may be understood as a smallest unit of the model segmentation. For example, as shown in FIG. 11, which is an exemplary schematic diagram illustrating an exemplary structure of a Willis ring according to some embodiments of the present disclosure, the arrows in the diagram illustrate a blood flow direction, and the dashed line illustrates a schematic illustration of segmenting the Willis ring in terms of the basic sub-models.

In some embodiments, the basic sub-models have different structures, such as a Y-shaped structure, an inverted Y-shaped structure, and a single vessel section (e.g., a straight vessel section, or a vessel section that has a certain curvature but is generally similar to a straight line).

In some embodiments, the basic sub-model includes a Y-shaped structure or an inverted Y-shaped structure.

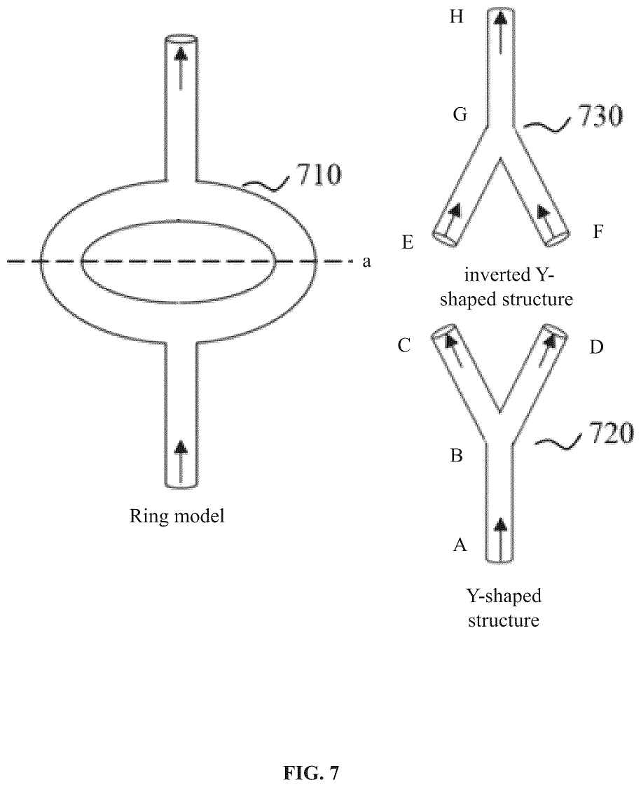

The Y-shaped structure refers to a configuration where a single vessel branches into two separate vessels at a branch point, forming a shape similar to the letter “Y”. Above the branch point, a vessel is connected to the branch point, and below the branch point, two or more vessels are connected to the branch point. When there are only two vessels below the branch point, the basic sub-model is similar to a “Y”. In some embodiments, the basic sub-model with a Y-shaped structure may be as shown in 720 of FIG. 7.

As used herein, “above” or “below” the branch point is determined by the blood flow direction. In the basic sub-model with a Y-shaped structure, the location through which blood flows first is above the branch point. Then, the blood flows through the branch point. The location through which the blood flows after flowing through the branch point is below the branch point. In the basic sub-model with a Y-shaped structure, blood flow bifurcates after passing through the branch point.

As shown in 720 of FIG. 7, the basic sub-model with a Y-shaped structure includes 4 nodes (denoted as A, B, C, and D) and 3 edges (denoted as AB, BC, and BD), and the blood flow direction is that blood first flows from A to B, bifurcates at B, and then flows from B to C and from B to D.

The inverted Y-shaped structure refers to a configuration where two separate vessels converge into a single vessel at a confluence point, forming a shape similar to an upside-down letter “Y”. Above the confluence point, two or more vessels are connected to the confluence point, and below the confluence point, a vessel is connected to the confluence point. When there are only two vessels above the confluence point, the basic sub-model is shaped like a “Y.” In some embodiments, the basic sub-model with an inverted Y-shaped structure may be as shown in 730 of FIG. 7.

As used herein, “above” or “below” the confluence point is determined by the blood flow direction. In the basic sub-model with an inverted Y-shaped structure, the location through which blood flows first is above the confluence point. Then, the blood flows through the confluence point. The location through which the blood flows after flowing through the confluence point is below the confluence point. In the basic sub-model with an inverted Y-shaped structure, blood from two vessels meets at the confluence point, and then flows together into a single vessel.

As shown in 730 of FIG. 7, the basic sub-model with an inverted Y-shaped structure includes 4 nodes (denoted as E, F, G, and H) and 3 edges (denoted as EG, FG, and GH), and the blood flow direction is that a blood stream flows from E to G, another blood stream flows from F to G, and the two blood streams meet at G and then flow together from G to H.

The composite sub-model refers to a sub-model that has a composite structure. In some embodiments, the composite sub-model is any combination of two or more basic sub-models. For example, the composite sub-model may include a ring model, an H-shaped model, etc., and the structure of the composite sub-model may be obtained by combining two or more of the basic sub-models.

The ring model refers to a sub-model with a closed structure. For example, the ring model may include three vessel sections including two single-vessel sections and one ring vessel section, and the two single-vessel sections are connected at different locations (e.g., top and bottom, or left and right, etc.) of the ring vessel section. In some embodiments, the ring model may be as shown in 710 of FIG. 7.

The H-shaped model includes two nodes, and its shape is similar to the letter “H.” In the H-shaped model, a first vessel section and a second vessel section are connected by a third vessel section disposed between the first vessel section and the second vessel section, and two ends of the third vessel section are connected to the first vessel section and the second vessel section, respectively, to form the two nodes. For example, two vertical vessel sections are located on the left and right, a horizontal vessel section is located between the two vertical vessel sections, two endpoints of the horizontal vessel section are connected to the two vertical vessel sections, and connection points of the horizontal vessel section and the two vertical vessel sections are the two nodes. In some embodiments, the H-shaped model may be as shown in 910 of FIG. 9.

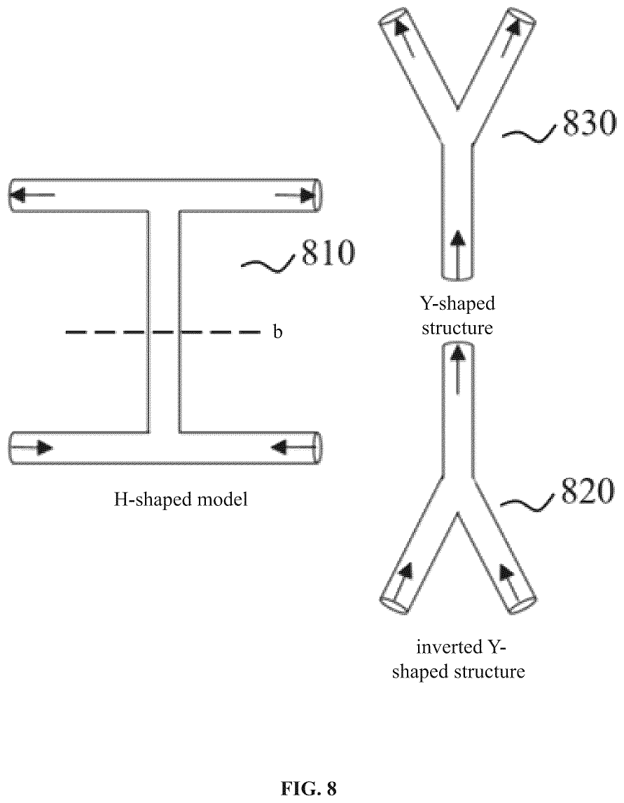

In some embodiments, as shown in 810 of FIG. 8, the H-shaped model may also be represented as two horizontal vessel sections located at the top and bottom and a vertical vessel section that is located between the two horizontal vessel sections and connected to the two horizontal vessel sections, respectively. Connection points of the vertical vessel section and the two horizontal vessel sections are the two nodes.

It should be noted that the basic sub-model with a Y-shaped structure, the basic sub-model with an inverted Y-shaped structure, the ring model, and the H-shaped model described above are generalized descriptions of the shapes and contours of the basic sub-models and the composite sub-models, i.e., which are only structured or contoured to have certain similarity, and are not intended to limit their contours or structures to have a canonical shape structure.

In some embodiments, the basic sub-model may be obtained by segmenting a composite sub-model. Specifically, the basic sub-model with an inverted Y-shaped structure and the basic sub-model with a Y-shaped structure may be obtained by segmenting various types of the composite sub-models, for example, as illustrated in FIG. 7, the ring model 710 may be segmented along the dashed line a to obtain the basic sub-model with an inverted Y-shaped structure 730 and the basic sub-model with a Y-shaped structure 720. As another example, as shown in FIG. 8, the H-shaped model 810 may be segmented along the dashed line b to obtain the basic sub-model with an inverted Y-shaped structure 820 and the basic sub-model with a Y-shaped structure 830. As a further example, as shown in FIG. 9, the H-shaped model 910 may be segmented along the dashed line c. Depending on the determination manner of the blood flow direction during the segmenting, the H-shaped model 910 may be segmented into two basic sub-models with Y-shaped structure, as shown in 920 of FIG. 9, or one basic sub-model with a Y-shaped structure and one basic sub-model with an inverted Y-shaped structure, as shown in 930 of FIG. 9.

In some embodiments, the processor may segment the reticular vessel model a plurality of times. For example, the reticular vessel model may be first segmented into a plurality of composite sub-models, and then the plurality of composite sub-models may be segmented into a plurality of basic sub-models.

In some embodiments, the processor may segment the reticular vessel model along the blood flow direction starting from at least one inlet of the reticular vessel model, and sequentially obtain the plurality of vessel sub-models. The inlet of the reticular vessel model refers to a node where blood flows into the reticular vessel model. For example, the reticular vessel model of the brain has four inlets, which are connection locations of the reticular vessel model of the brain with the right and left internal carotid arteries, and the right and left vertebral arteries. As another example, the reticular vessel model of the liver has 2 inlets, which are connection locations of the reticular vessel model of the liver with the portal vein and the hepatic artery. The processor may determine the inlet of the reticular vessel model in a variety of ways, e.g., image recognition.

The processor may determine nodes corresponding to branch points and confluence points in the reticular vessel model as first class nodes, and determine any point on a vessel section between two adjacent first class nodes as a second class node. Starting from at least one inlet of the reticular vessel model along the blood flow direction, if encountering a branch point in the first class nodes, the processor may determine a basic sub-model with a Y-shaped structure by selecting the branch point, one node (e.g., an inlet or a second class node) that is adjacent to and located in a relative pre-order (upstream) of the branch point, and at least two nodes (e.g., outlets or second class nodes) that are adjacent to and located in a relative post-order (downstream) of the branch point, and vessel sections between the selected nodes.

If the confluence point in the first class nodes is encountered, the processor may determine a basic sub-model with an inverted Y-shaped structure by selecting the confluence point, at least two nodes (e.g., inlets or second class nodes) that are adjacent to and located in a relative pre-order (upstream) of the confluence point, and one node (e.g., an outlet or a second class node) that is adjacent to and located in a relative post-order (downstream) of the confluence point, and vessel sections between the selected nodes.

The processor may set the blood flow direction of each vessel section in each basic model when performing the segmentation. A vessel section may be labeled with two opposite blood flow directions. More details regarding processing the vessel section with two opposite blood flow directions may be found in description regarding processing a collision flow.

In some embodiments, the processor may segment a composite sub-model including a plurality of nodes to obtain a plurality of basic sub-models.

In 304, connections of the plurality of vessel sub-models may be determined.

The connections refer to ways in which the vessel sub-models are connected to each other. For example, there is a connection between a vessel sub-model I and a vessel sub-model II. The vessel sub-model I has a plurality of nodes, such as a₁, a₂, and a₃, and the vessel sub-model II also has a plurality of nodes, such as b₁, b₂, and b₃, and the connection may be represented as: a₁ is connected to b₂, or a₂ is connected to b₃, etc.

In some embodiments, the connections reflect at least one relative pre-order relationship and at least one relative post-order relationship of the plurality of vessel sub-models.

Pre-order and post-order are relative concepts. For example, assuming that blood flows through the vessel sub-model I, the vessel sub-model II, and a vessel sub-model III sequentially. The vessel sub-model through which blood flows first corresponds to the relative pre-order, and the vessel sub-model through which blood flows later corresponds to the relative post-order. For the vessel sub-model I and the vessel sub-model II, the vessel sub-model I corresponds to the relative pre-order and the vessel sub-model II corresponds to the relative post-order, and for the vessel sub-model II and the vessel sub-model III, the vessel sub-model II corresponds to the relative pre-order and the vessel sub-model III corresponds to the relative post-order.

In some embodiments, the connections may include at least one blood flow direction of the plurality of vessel sub-models and at least one correspondence of vessel branches of the plurality of vessel sub-models.

The blood flow direction may reflect an inflow and an outflow of blood as it flows through a blood vessel. For example, denoting two endpoints of a blood vessel section by points a and b, respectively, the blood flow direction may be a to b, indicating that blood flows into the blood vessel section from point a and flows out of the vessel section from point b.

In some embodiments, the processor may determine the relative pre-order and relative post-order relationship of the vessel sub-models through the blood flow direction. For example, the processor may determine, based on the blood flow direction, the vessel sub-model that the blood flowing out as a relative pre-order sub-model, and the vessel sub-model that the blood flowing into as a relative post-order sub-model.

In some embodiments, the processor may determine the blood flow direction of each vessel sub-model in a variety of ways. For example, the processor may determine the blood flow direction of each vessel sub-model along the blood flow direction from the entrance of the reticular vessel model. As another example, the processor may reversely infer the blood flow direction of each vessel sub-model from the exit of the reticular vessel model.

In some embodiments, the determining the blood flow parameter of the reticular vessel model includes: determining, based on physiological data of the object and the connections of the plurality of vessel sub-models, the blood flow parameter of the reticular vessel model.

FIG. 4 is an exemplary flowchart illustrating an exemplary for coupling processing according to some embodiments of the present disclosure. In some embodiments, process 400 may be performed by a system for determining a blood flow parameter of a reticular vessel or a processor (e.g., the processor 140). As shown in FIG. 4, the process 400 includes the following operations.

In 402, based on physiological data of an object, a blood flow parameter of a relative pre-order sub-model of a plurality of vessel sub-models may be determined.

In some embodiments, the blood flow parameter of a relative pre-order sub-model of a plurality of vessel sub-models is an outlet blood flow parameter of the relative pre-order sub-model.

The physiological data of the object refers to quantitative measurement data regarding a physical condition and a function of the object. For example, the physiological data may include one or more of a blood pressure, a heart rate, a body temperature, a respiration rate, an oxygen saturation, a Doppler ultrasound blood flow velocity, etc. The physiological data may be used to assess a health status, diagnose diseases, and monitor the effectiveness of treatments of the object (e.g., a patient).

For two vessel sub-models, the relative pre-order sub-model refers to a vessel sub-model through which the blood flows first. For example, for vessel sub-model I, vessel sub-model II, and vessel sub-model III described above, blood flows in the order I->II->III, then vessel sub-model I is the relative pre-order sub-model of vessel sub-model II, and vessel sub-model II is the relative pre-order sub-model of vessel sub-model III. As another example, as shown in FIG. 10, in 1010, a first H-shaped model is segmented into a basic sub-model with a Y-shaped structure and a basic sub-model with an inverted Y-shaped structure, and the basic sub-model with an inverted Y-shaped structure is the relative pre-order sub-model of the basic sub-model with a Y-shaped structure based on the blood flow direction.

It should be noted that relative pre-order and relative post-order are concepts between the basic sub-models, between the composite sub-models, or between the basic sub-models and the composite sub-models. For example, referring to FIG. 10, assuming that blood flows from r1 and r2 of the first H-shaped model 1010 and then flows out through c₂ to d₁ into a second H-shaped model, i.e., there is a connection between c₂ and d₁. Then the relative pre-order and relative post-order may be a relationship of the first H-shaped model and the second H-shaped model (as shown by the dashed line a in FIG. 10), or between the first H-shaped model and the basic sub-model with a Y-shaped structure obtained by segmenting the second H-shaped model (as shown by the dashed line b in FIG. 10), or between the basic sub-model with a Y-shaped structure obtained by segmenting the first H-shaped model and the second H-shaped model (as shown by the dashed line c in FIG. 10), or between the basic sub-model with a Y-shaped structure obtained by segmenting the first H-shaped model and the basic sub-model with a Y-shaped structure obtained by segmenting the second H-shaped model (as shown by the dashed line d in FIG. 10).

In some embodiments, the processor may use computational fluid dynamics methodology to determine the blood flow parameter of the relative pre-order sub-model. For example, the determination process may be as shown in the embodiments below.

The processor may perform meshing on the relative pre-order sub-model to generate a meshed three-dimensional vessel model; and determine, based on the relevant physiological data of the object (or empirical or reference values) and the meshed three-dimensional vessel model, a boundary parameter of the relative pre-order sub-model. The boundary parameter may include at least one of a blood flow velocity, a blood flow volume, a blood pressure, a distal vessel resistance, and a ratio of the blood pressure and the distal vessel resistance at an outlet of the relative pre-order sub-model, and at least one of a blood flow velocity, a blood flow volume, and a blood pressure at an inlet of the relative pre-order sub-model. The processor may determine a flow field distribution of the meshed three-dimensional vessel model using computational fluid dynamics based on the boundary parameter of the relative pre-order sub-model.

In some embodiments, the processor may mesh the relative pre-order sub-model by various manners, such as Delaunay triangulation. In some embodiments, the processor may determine the boundary parameter by various manners, such as using machine learning models.

In 404, a blood flow parameter of a relative post-order sub-model of the plurality of vessel sub-models may be determined by performing, based on the connection and the blood flow parameter of the relative pre-order sub-model.

In some embodiments, when determining the blood flow parameters of the relative post-order sub-model based on the connection and the blood flow parameters of the relative pre-order sub-model, the boundary parameters of the relative post-order sub-model are determined based on the outlet blood flow parameters of the relative pre-order sub-model and the connection. Based on the boundary parameters of the relative post-order sub-model, the blood flow parameters of the relative post-order sub-model are determined. Details regarding determining the boundary parameters of the relative post-order sub-model based on the outlet blood flow parameters of the relative pre-order sub-model and the connection can be found elsewhere in the present disclosure (e.g., description in connection with operation 404). Details regarding determining all the blood flow parameters of the relative post-order sub-model based on the boundary parameters of the relative post-order sub-model can be found elsewhere in the present disclosure (e.g., description in connection with operation 404). In some embodiments, the boundary parameters of vessel sub-models that do not have a relative pre-order sub-model (e.g., a vessel sub-model of which the inlet is the inlet of the reticular vessel model) can be determined by Doppler ultrasound or MRI.

For two vessel sub-models, the relative post-order sub-model refers to a vessel sub-model through which blood flows later. For example, for vessel sub-model I, vessel sub-model II, and vessel sub-model III described above, blood flows in the order I->II->III, then vessel sub-model II is the relative post-order sub-model of vessel sub-model I, and vessel sub-model III is the relative post-order sub-model of vessel sub-model II. As another example, in 1010, the basic sub-model with a Y-shaped structure is the relative post-order sub-model of the basic sub-model with an inverted Y-shaped structure.

In some embodiments, the processor may determine, based on the connection and the blood flow parameter of the relative pre-order sub-model, a boundary parameter of the relative post-order sub-model; and based on the boundary parameter of the relative post-order sub-model, determine the blood flow parameter of the relative post-order sub-model.

In some embodiments, the boundary parameters refer to the blood flow parameter at the inlet and/or the outlet of the plurality of vessel sub-models.

The boundary parameter includes one or more of a blood flow direction, a blood flow pressure, and a blood flow rate of the inlet of the relative post-order sub-model.

In some embodiments, the boundary parameter includes one or more of a blood flow direction, a blood flow pressure, and a blood flow rate of the inlet of the relative post-order sub-model.

In some embodiments, the blood flow parameter of the relative pre-order model may be already known by means of computational fluid dynamics, and a boundary parameter (e.g., a blood flow pressure corresponding to the inlet) of the relative post-order sub-model may be determined based on the connection and the blood flow parameter of the relative pre-order model. Then, on the basis of the determined boundary parameter of the relative post-order sub-model, a blood flow parameter of the relative post-order model, including inlet and outlet blood flow parameters and the result of the overall 3D model, etc., is determined by computational fluid manners. For example, the processor may mesh the relative post-order sub-model to obtain a meshed three-dimensional vessel model of the relative post-order sub-model; and on the basis of the blood flow parameter of the relative pre-order sub-model, determine the boundary parameter of the relative post-order sub-model based on the blood flow parameter of the relative pre-order sub-model, the relevant physiological data of the object, and the corresponding meshed three-dimensional vessel model of the relative post-order sub-model. The boundary parameter may include at least one of a blood flow velocity, a blood flow volume, a blood pressure, a distal vessel resistance, and a ratio of the blood pressure and the distal vessel resistance at an outlet of the relative post-order sub-model, and at least one of a blood flow velocity, a blood flow volume, and a blood pressure at an inlet of the relative post-order sub-model. The processor may determine the flow field distribution of the meshed three-dimensional vessel model using computational fluid dynamics based on the boundary parameter of the relative post-order sub-model.

In some embodiments, the processor may determine the boundary parameter of the relative post-order sub-model based on a topology and vessel attributes (e.g., a vessel length, a thickness, etc.) of the relative post-order sub-model, the outlet blood flow parameter of the relative pre-order sub-model, and the relevant physiological data of the object.

In some embodiments, when the basic sub-model with a Y-shaped structure and the basic sub-model with an inverted Y-shaped structure are connected, the processor may determine the boundary parameter of the relative post-order sub-model (assumed to be the basic sub-model with an inverted Y-shaped structure) based on the connection and the blood flow parameter of the relative pre-order sub-model (assumed to be the basic sub-model with a Y-shaped structure).

In some embodiments, the processor may determine a plurality of candidate blood flow parameters of the relative post-order sub-model using a plurality of determination manners; and determine the blood flow parameter of the relative post-order sub-model by determining a weighted average of the plurality of candidate blood flow parameters. The weights may be preset.

For example, for more accurate determination manners, higher weights may be set. As another example, a lower weight may be set for simpler determination manners. The determination manners of the blood flow parameter are determination manners that are readily understandable and accessible to a person skilled in the art, and the present disclosure will not repeat herein.

Using the plurality of determination manners to determine the plurality of candidate blood flow parameters and taking the weighted average of the candidate blood flow parameters as the blood parameter of the vessel sub-model can reduce error. At the same time, setting different weights for the plurality of determination manners of the blood parameters can make the determination of the blood parameters more reasonable and improve the determination accuracy.

In some embodiments, the processor may divide the reticular vessel model into a plurality of basic sub-models and/or a plurality of composite sub-models. The processor may determine a count of the plurality of determination manners of the blood flow parameter of the relative post-order vessel sub-model in a plurality of ways. For example, the count of the plurality of determination manners may be preset based on actual needs. In some embodiments, the processor may obtain the count of the plurality of determination manners through historical data. For example, for a relative post-order vessel sub-model with one or more target sub-models, the processor may determine a corresponding historical relative post-order vessel sub-model with one or more historical target sub-models with the same count as the one or more target sub-models. The target sub-models are the relative pre-order sub-models corresponding to the relative post-order sub-model. The historical target sub-models are the relative pre-order sub-models corresponding to the historical relative post-order sub-model. The processor may designate a count of the plurality of determination manners of blood flow parameters of the historical relative post-order vessel sub-model as the count of the plurality of determination manners of the blood flow parameters of the relative post-order vessel sub-model.

In some embodiments, the processor may determine the count of the plurality of determination manners of the blood flow parameter based on the count of target sub-models. The more the count of the target sub-models, the more the count of the plurality of determination manners of the blood flow parameter.

In some embodiments, the processor may obtain the count of the plurality of determination manners of the blood flow parameter by setting a preset threshold sequence. The preset threshold sequence refers to a sequence that includes a plurality of sub-thresholds arranged in an order, e.g., an ascending or a descending order. Each of the plurality of sub-thresholds corresponds to a candidate count. In response to determining that the count of the target sub-models exceeds one of the plurality of sub-thresholds, the processor may determine the candidate count corresponding to the sub-threshold as the count of the plurality of determination manners of the relative post-order sub-model.

In some embodiments, when the count of target sub-models exceeds more than one of the plurality of sub-thresholds, in the more than one sub-model, the sub-threshold closest to the count of target sub-models is selected as the count of the plurality of determination manners of the relative post-order sub-model.

The sub-threshold is determined based on actual needs. In some embodiments, the sub-threshold may be determined based on the accuracy of the blood flow parameter determination performed by a current segmentation manner.

The higher the accuracy of the blood flow parameter determination, the more reasonable the corresponding candidate segmentation manner may be, and the sub-thresholds may be set relatively high, which may save computing resources while guaranteeing the accuracy of the blood flow parameter determination.

Blood flows through the target sub-models and then flows into the relative post-order vessel sub-model. Therefore, the greater the count of the target sub-models, the greater the cumulative error in the determination manner of the blood flow parameter of the corresponding relative post-order vessel sub-model. Thus, using the plurality of determination manners to determine the blood flow parameters based on the count of the target sub-models may reduce error. In some embodiments, the blood flow parameters of the relative pre-order sub-model and the relative post-order sub-model may also be determined by means of machine learning. For example, the physiological data of the object and the relative pre-order sub-model may be input into a first deep learning model, and the first deep learning model may output the blood flow parameter of the relative pre-order sub-model, and then the blood flow parameters of the relative pre-order sub-model, the physiological data of the object, and the relative post-order sub-model may be input into a second deep learning model, and the second deep learning model may output the blood flow parameter of the relative post-order sub-model.

In some embodiments, the processor may obtain the first deep learning model and the second deep learning model by joint training based on the labeled training samples, wherein the training samples may include sample physiological data of a sample object, a sample relative pre-order sub-model, and a sample relative post-order sub-model. The label may be an actual blood flow parameter of the sample relative pre-order sub-model and an actual blood flow parameter of the sample relative post-order sub-model.

In some embodiments, the process of joint training may include: inputting the sample physiological data of the sample object and the sample relative pre-order sub-model into the initial first deep learning model, obtaining the blood flow parameters of the sample relative pre-order sub-model output by the initial first deep learning model, inputting the blood flow parameters of the sample relative pre-order sub-model, the sample physiological data of the sample object, and the sample relative post-order sub-model into the initial second deep learning model, obtaining the blood flow parameters of the sample relative post-order sub-model output by the initial second deep learning model, determining a first loss function based on actual blood flow parameters of the sample relative pre-order sub-model and the blood flow parameters output by the first deep learning model, determining a second loss function based on actual blood flow parameters of the sample relative post-order sub-model and the blood flow parameters output by the second deep learning model, and synchronously updating parameters of the first deep learning model and the second deep learning model based on the first loss function and the second loss function. The trained first deep learning model and the trained second deep learning model may be obtained by parameter update. In some embodiments, the first deep learning model may be updated based on the first loss function and the second loss function, and the parameters of the second deep learning model may be updated based on the second loss function.

Training efficiency may be improved by joint training the first deep learning model and the second deep learning model.

It should be noted that the above exemplary determination manners of the blood flow parameters are only for exemplary purposes, and a person of skill in the art may use various determination manners of the blood flow parameters to calculate the blood flow parameters of the relative pre-order sub-model and the relative post-order sub-model.

After determining the blood flow parameters for all the vessel sub-models, the blood flow parameter for the reticular vessel model may be determined. For example, the blood flow parameter for the reticular vessel model may include the blood flow parameters for all the vessel sub-models.

In some embodiments, the determining the blood flow parameter of the reticular vessel model by performing, based on the plurality of vessel sub-models, coupling processing may further include: modifying a boundary parameter of at least one of the plurality of vessel sub-models corresponding to a collision flow; and determining a modified blood flow parameter of the at least one of the plurality of vessel sub-models by re-performing, based on the modified boundary parameter, the coupling processing. The blood vessel of the one of the plurality of vessel sub-models corresponds to a collision flow.

FIG. 5 is an exemplary flowchart illustrating a process for coupling processing according to some other embodiments of the present disclosure. In some embodiments, process 500 may be performed by a system for determining a blood flow parameter of a reticular vessel or a processor (e.g., the processor 140). As shown in FIG. 5, the process 500 includes the following operations.

In 502, whether there is a collision flow in the plurality of vessel sub-models may be determined.

In some embodiments, the processor may determine whether there is a collision flow in the plurality of vessel sub-models when the at least one blood flow direction of the plurality of vessel sub-models and the at least one correspondence of vessel branches of the plurality of vessel sub-models are determined.

In the model partitioning, the blood flow direction of each of the plurality of vessel sub-models may be determined. If the determined blood flow direction of each of the plurality of vessel sub-models is not optimal, the accuracy of the subsequent blood flow parameter determination of the reticular vessel model may be affected. Therefore, after determining the blood flow direction of each of the plurality of vessel sub-models, the processor may determine whether there is a collision flow in the plurality of vessel sub-models to determine whether the determined blood flow direction of each of the plurality of vessel sub-models is optimal.

In some embodiments, the processor may determine whether there is a collision flow in one vessel sub-model, and/or between two vessel sub-models.

The collision flow refers to a situation where two opposite blood flow directions are marked in a blood vessel without a branch point and a confluence point in one vessel sub-model.

In some embodiments, the collision flow is also usually seen at a connection location of different vessel sub-models. For example, a vessel section OA in vessel sub-model I is connected to a vessel section OB in vessel sub-model II at connection point O that is neither a branch point nor a confluence point. For the vessel section OA, the blood flow direction is A->O, and for the vessel section OB, the blood flow direction is B->O. There is a blood collision at the connection location O, which is referred to as a collision flow.

In a vessel sub-model, there may be more than one manner for the blood flow direction of the vessel sub-model. In the process for determining the blood flow direction of the vessel sub-model, one of the more than one manner for the blood flow direction may be selected as the blood flow direction of the vessel sub-model. The processor may determine whether the selected manner is optimal. If the selected manner is not optimal (e.g., there is a collision flow corresponding to the vessel sub-model), the processor may modify the blood flow direction of the vessel sub-model.

For example, referring to FIG. 10, in 1010, according to the blood flow direction in the first H-shaped model (shown by the arrows), it may be determined that the blood flows into the blood vessel through the endpoints r₁ and r₂, flows through the point f, and then flows out through the endpoints c₁ and c₂. At a point f of the first H-shaped model, the first H-shaped model is segmented into a basic sub-model with an inverted Y-shaped structure and a basic sub-model with a Y-shaped structure, and the segmentation point is f. After the segmentation, the blood flow direction is first from r₁ and r₂ to f₁, and then from f₂ (f₁ and f₂ may actually be considered as the same point and correspond to point f) to c₁ and c₂ to flow out. In this case, there is only one manner of the blood flow direction in the first H-shaped model, there is no collision flow in the first H-shaped model, and there is no collision flow between the basic sub-model with an inverted Y-shaped structure and the basic sub-model with a Y-shaped structure.

If there is no collision flow, the blood flow parameter of the relative post-order sub-model may be determined directly based on the blood flow parameter of the relative pre-order sub-model.

Referring to FIG. 10, in 1020, blood flows into the second H-shaped model from points d₁ and d₂, and flows out of the second H-shaped model from points e₁ and e₂. There are two manners of the blood flow directions of the second H-shaped model. The first manner is that blood bifurcates at the two nodes of the second H-shaped model (as shown in FIG. 12). The second manner is that blood bifurcates at only one node of the second H-shaped model and at the other node, blood does not flow through point g. The first manner may cause a collision flow at point g. The second manner may not cause a collision flow in the second H-shaped model. At the point g of the second H-shaped model, the second H-shaped model is segmented. The segmentation includes two types according to the two manner of the blood flow direction. One type is to segment the second H-shaped model into two basic sub-models with Y-shaped structure, which corresponds to the first manner. In this type, the blood flows from d₁ to g₁ and from d₂ to g₂ (g₁ and g₂ may actually be considered as the same point and correspond to point g). There is a collision flow between the two basic sub-models with Y-shaped structure. The other type is to segment the second H-shaped model into one basic sub-model with a Y-shaped structure and one basic sub-model with an inverted Y-shaped structure, which corresponds to the second manner. In this type, the blood flows from d₁ to g₁ and from g₂ to e₂. There is no collision flow between the basic sub-model with a Y-shaped structure and the basic sub-model with an inverted Y-shaped structure.

More details regarding determining the blood flow direction of the collision flow may be found in other contents of the present disclosure (e.g., FIG. 3).

In some embodiments, if blood from different vessel sub-models flows to the same connection location of the different vessel sub-models, the processor may determine that there is a collision flow between the different vessel sub-models.

In 504, if there is a collision flow in the plurality of vessel sub-models, a boundary parameter of at least one of the plurality of vessel sub-models corresponding to the collision flow may be modified.

In some embodiments, the blood vessel of the vessel sub-model corresponding to the collision flow refers to a blood vessel in which a collision flow occurs.

In some embodiments, the modification of the boundary parameter includes modifying one or more of a blood flow direction, a blood flow pressure, a blood flow rate, a blood flow velocity, a blood flow resistance, or the like, of the blood flow at an inlet and an outlet of the blood vessel. For example, the blood flow from b₂ to b₁ may be modified to from b₁ to b₂ to eliminate the collision flow. In some embodiments, in addition to modifying the blood flow direction, one or more of the blood flow pressure, the blood flow rate, the blood flow velocity, the blood resistance, etc., may be modified. For example, after modifying the blood flow direction, information such as the blood flow pressure, the blood flow rate, the blood flow velocity, and the blood flow resistance is modified accordingly.

In some embodiments, the boundary parameter of any one of the vessel sub-models corresponding to the collision flow may be modified based on a current progress of blood flow parameter determination. For example, for vessel sub-models I, II, III, and IV, assuming that there is a collision flow between vessel sub-models I and II, as it is uncertain whether the blood flow direction of vessel sub-model I is correct or the blood flow direction of vessel sub-model II is correct, the blood flow direction in either vessel sub-model I or vessel sub-model II may be modified. As another example, assuming that there is a collision flow between vessel sub-models Il and III, if it has been determined that the blood flow direction within vessel sub-model II is correct, the blood flow direction in vessel sub-model III may be modified.

In some embodiments, referring to FIG. 12, the processor may label the two vessel sub-models between which a collision flow occurs as a first sub-model and a second sub-model, respectively.

The processor may correct the blood flow direction of the first sub-model or the second sub-model. The processor may also determine, based on the corrected blood flow direction, a relative pre-order sub-model (assumed to be the first sub-model) in the first sub-model and the second sub-model, and determine, based on the connection and the blood flow parameter of the relative pre-order sub-model, a blood flow parameter of a relative post-order sub-model (assumed to be the second sub-model). The manner of determining the blood flow parameter may be described in connection with the operation 208 and related description thereof; and the manner of determining the blood flow parameter of the relative post-order sub-model may be described in connection with FIG. 4 and related description thereof.

In some embodiments, as shown in FIG. 12, the first sub-model (the sub-model on the left) includes 4 nodes (denoted as A, F, B, and C₁) and 3 edges (denoted as AF, FB, and FC₁), and the blood flow direction is from A to F, from F to B, and from F to C₁. The second sub-model (the sub-model on the right) includes 4 nodes (denoted as D, G, C₂, and E) and 3 edges (denoted as DG, GC₂, and GE), and the blood flow direction is from D to G, from G to C₂ and from G to E. It is clear that FC₁ in the first sub-model and GC₂ in the second sub-model are located in the same vessel section, but their blood flows are in the opposite direction, that is, there is a collision flow. The node C₁ and the node C₂ are two endpoints obtained by segmenting a vessel section at a certain point.

For the first sub-model, the processor may determine, based on the blood flow parameter of node A, blood flow parameters of the node B, the node F, and the node C₁. For the second sub-model, the processor may determine, based on the blood flow parameter of the node D, blood flow parameters of the node C₂, the node G, and the node E. More details regarding the determination manner of blood flow parameter may be found in other contents of the present disclosure (e.g., FIG. 4). The processor may compare the blood flow volume of the two vessel sections (FC₁, GC₂, respectively) where the collision flow occurs, i.e., compare the blood flow volume of the node C₁ and the node C₂, and use the blood flow direction of the vessel section with the larger blood flow volume (assumed to be the FC₁) as the final corrected blood flow direction of the two vessel sections. That is, the blood flow direction of the GC₂ is corrected from the original direction from G to C₂ to a direction from C₂ to G. The processor may use the first sub-model as the relative pre-order sub-model and the second sub-model as the relative post-order sub-model based on the blood flow direction.

In 506, a modified blood flow parameter of the at least one of the plurality of vessel sub-models may be determined by re-performing, based on the modified boundary parameter, the coupling processing.

Referring to FIG. 12, before modifying the blood flow direction, for the first sub-model, the processor may determine, based on the blood flow parameter of node A, blood flow parameters of the node B and the node C₁. The node A of the first sub-model may either be the inlet of the reticular vessel model or the exit of at least one relative pre-order sub-model located upstream of node A. The blood flow parameters at node A are always known in advance (e.g., determined using techniques like ultrasound or MRI) or can be calculated based on the blood flow parameters at the outlet of the at least one relative pre-order sub-model located upstream of node A. For the second sub-model, the processor may determine, based on the blood flow parameter of the node D, blood flow parameters of the node C₂ and the node E. More details regarding the determination manner of blood flow parameter may be found in other contents of the present disclosure (e.g., FIG. 4).

After modifying the blood flow direction, the processor may determine the blood flow parameters of the node B and the node C₁ based on the blood flow parameter of the node A. The processor may determine the blood flow parameter of the node E based on the blood flow parameter of the node C₁, the blood flow parameter of the node D, and the connection, by performing the coupling process. More details regarding the blood flow parameter and the coupling processing may be found in other contents of the present disclosure (e.g., FIG. 4). In some embodiments, the processor may also take another node C₃ (not shown in FIG. 12) on the vessel section FG, and the node C₃ is different from the nodes C₁ and C₂. The processor may determine the blood flow parameter of the node C₃ and redetermine the blood flow parameter of the node B based on the blood flow parameters of the node A, the node C₁, and the node C₂, and a connection. The processor may determine the blood flow parameter of the node E by performing the coupling processing based on the blood flow parameters of the node C₃ and the node D, and the connection. More details regarding the blood flow parameter and the coupling processing may be found in other contents of the present disclosure (e.g., FIG. 4).

The coupling processing after modifying the boundary parameter is the same as the determination manner the blood flow parameter of described in FIG. 4 and FIG. 5, and the detailed description may be found in the previous description, which is not repeated here.

In some embodiments, after modifying the boundary parameters of one of the two vessel sub-models corresponding to the collision flow, the boundary parameters of the post-order sub-models relative to the two vessel sub-models corresponding to the collision flow also need to be adaptively adjusted. More details regarding how to modify the boundary parameters of the post-order sub-models may be found in other contents of the present disclosure (e.g., FIG. 4).

It should be noted that the foregoing descriptions of the respective processes are for the purpose of exemplification and illustration only and do not limit the scope of application of the present disclosure. For a person skilled in the art, various corrections and changes may be made to the respective processes under the guidance of the present disclosure. However, these corrections and changes remain within the scope of the present disclosure. For example, adding a storage operation or the like.

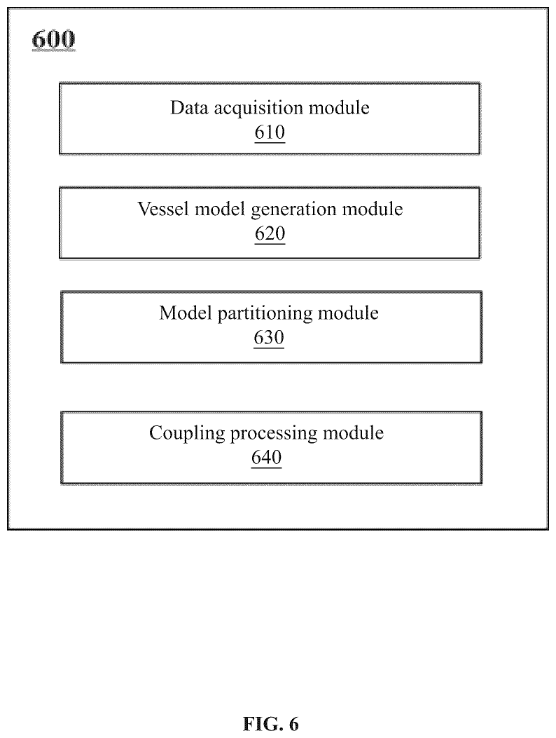

FIG. 6 is an exemplary block diagram illustrating a system for determining a blood flow parameter of a reticular vessel according to some embodiments of the present disclosure. As shown in FIG. 6, a system 600 may include the data acquisition module 610, the vessel model generation module 620, the model partitioning module 630, and the coupling processing module 640.

The data acquisition module 610 may be configured to obtain a vessel image of an object.

The vessel model generation module 620 may be configured to generate a reticular vessel model by performing, based on the vessel image, a vessel segmentation.

The model partitioning module 630 may be configured to generate, by performing model partitioning based on the reticular vessel model, a plurality of vessel sub-models.

The coupling processing module 640 may be configured to determine the blood flow parameter of the reticular vessel model by performing, based on the plurality of vessel sub-models, coupling processing.

More details regarding the modules shown in FIG. 6 may be found in the relevant flowcharts of the present disclosure, i.e., the relevant descriptions of FIG. 2 to FIG. 5.

It should be appreciated that the system 600 and modules thereof illustrated in FIG. 6 may be implemented utilizing a variety of means. For example, in some embodiments, the system 600 and the modules thereof may be implemented by hardware, software, or a combination thereof. The hardware portion may be implemented utilizing specialized logic; the software portion may be stored in a memory and executed by an appropriate instruction execution system, e.g., a microprocessor or specially designed hardware. Those skilled in the art will appreciate that the methods and systems described above may be implemented using computer executable instructions and/or included in processor control code, for example, provided on carrier media, e.g., disks, CDs, or DVD-ROMs, programmable memory such as read-only memories (firmware), or data carriers such as optical or electronic signal carriers. The system and modules thereof of the present disclosure may be realized not only with hardware circuits such as ultra-large scale integrated circuits or gate arrays, semiconductors such as logic chips, transistors, etc., or programmable hardware devices such as field-programmable gate arrays, programmable logic devices, etc., but also with software executed, for example, by processors of various types, or with a combination of the above hardware circuits and software (e.g., firmware).

It is to be noted that the above description of the system for determining a blood flow parameter of a reticular vessel and the modules thereof is provided only for descriptive convenience, and does not limit the present disclosure to the scope of the embodiments cited. It is to be understood that for a person skilled in the art; after understanding the principle of the system, it may be possible to arbitrarily combine the individual modules or form a sub-system to be connected to the other modules without departing from the principle. In some embodiments, the data acquisition module 610, the vessel model generation module 620, the model partitioning module 630, and the coupling processing module 640 disclosed in FIG. 6 may be different modules in a system module, or may be a single module that realizes the functions of two or more of the above-described modules. For example, the individual modules may share a common storage module, and the individual modules may each have a respective storage module. Morphs such as these are within the scope of protection of the present disclosure.

Having thus described the basic concepts, it may be rather apparent to those skilled in the art after reading this detailed disclosure that the foregoing detailed disclosure is intended to be presented Merely by way of example and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

In addition, unless clearly stated in the claim, the order of processing element and sequence described in the present disclosure, the use of alphanumeric characters or the use of other names are not used to limit the order of the process and method of the present disclosure. Although some currently useful invention embodiments are considered to be discussed by various examples in the above disclosure, it should be understood that such details only serve the purpose of illustration, and additional claims are not limited to the disclosed embodiments. On the contrary, claims are intended to cover all amendments and equivalent combinations that meet the essence and scope of the present disclosure. For example, although the system components described above can be realized by hardware devices, it is also possible to only realize by the solution of software, such as installing the described system on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required characteristics of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

1. A method for determining a blood flow parameter of a reticular vessel, implemented on a device including at least one processing device and at least one storage device, the method comprising:
obtaining vessel data of an object;
generating a reticular vessel model by performing, based on the vessel data, vessel segmentation;
generating, by performing model partitioning based on the reticular vessel model, a plurality of vessel sub-models; and
determining the blood flow parameter of the reticular vessel model by performing, based on the plurality of vessel sub-models, coupling processing.

2. The method of claim 1, wherein the generating, by performing model partitioning based on the reticular vessel model, a plurality of vessel sub-models includes:
segmenting the reticular vessel model into the plurality of vessel sub-models; and
determining connections of the plurality of vessel sub-models.

3. The method of claim 2, wherein the determining the blood flow parameter of the reticular vessel model includes:
determining, based on physiological data of the object and the connections of the plurality of vessel sub-models, the blood flow parameter of the reticular vessel model.

4. The method of claim 2, wherein the connections are related to at least one blood flow direction of the plurality of vessel sub-models and at least one correspondence of vessel branches of the plurality of vessel sub-models.

5. The method of claim 2, wherein the segmenting the reticular vessel model into the plurality of vessel sub-models includes:
generating a plurality of candidate segmentation manners;
for each of the plurality of candidate segmentation manners, predicting, through a prediction model, an accuracy of blood flow parameter determination performed using the candidate segmentation manner; and
segmenting the reticular vessel model into the plurality of vessel sub-models based on the candidate segmentation manner corresponding to a highest accuracy.

6. The method of claim 2, wherein the segmenting the reticular vessel model into the plurality of vessel sub-models includes
determining a segmentation refining degree of the reticular vessel model; and
segmenting the reticular vessel model based on the segmentation refining degree.

7. The method of claim 1, wherein the determining the blood flow parameter of the reticular vessel model by performing, based on the plurality of vessel sub-models, coupling processing includes:
determining, based on physiological data of the object, a blood flow parameter of a relative pre-order sub-model of the plurality of vessel sub-models; and
determining a blood flow parameter of a relative post-order sub-model of the plurality of vessel sub-models based on a connection and the blood flow parameter of the relative pre-order sub-model.

8. The method of claim 7, wherein determining the blood flow parameter of the relative post-order sub-model includes:
determining a plurality of candidate blood flow parameters of the relative post-order sub-model using a plurality of determination manners; and
determining the blood flow parameter of the relative post-order sub-model by determining a weighted average of the plurality of candidate blood flow parameters.

9. (canceled)

10. The method of claim 7, wherein the determining a blood flow parameter of a relative post-order sub-model of the plurality of vessel sub-models by performing, based on a connection and the blood flow parameter of the relative pre-order sub-model includes:
determining, based on the connection and the blood flow parameter of the relative pre-order sub-model, a boundary parameter of the relative post-order sub-model; and
determining, based on the boundary parameter of the relative post-order sub-model, the blood flow parameter of the relative post-order sub-model.

11. The method of claim 1, wherein the determining the blood flow parameter of the reticular vessel model by performing, based on the plurality of vessel sub-models, coupling processing includes:
modifying a boundary parameter of at least one of the plurality of vessel sub-models corresponding to a collision flow; and
determining a modified blood flow parameter of the at least one of the plurality of vessel sub-models by re-performing, based on the modified boundary parameter, the coupling processing.

12. The method of claim 11, wherein before modifying a boundary parameter of at least one of the plurality of vessel sub-models corresponding to a collision flow, the method further comprising:
determining whether there is a collision flow in the plurality of vessel sub-models based on the blood flow direction of the plurality of vessel sub-models.

13. The method of claim 1, wherein the determining the blood flow parameter of the reticular vessel model by performing, based on the plurality of vessel sub-models, coupling processing further includes:
determining a plurality of blood flow parameters of the vessel sub-model in a specific region each of which is determined using one of a plurality of determination manners, wherein the specific region includes a Willis ring region, a lesion of the object, a region within a preset range of the lesion, or a region with a collision flow; and
determining a final blood flow parameter of the vessel sub-model based on the plurality of blood flow parameters caused by the plurality of determination manners.

14. The method of claim 13, comprising:
determining the blood flow parameter of the vessel sub-model in the specific region using a trained machine learning model.

15. The method of claim 1, wherein the reticular vessel includes a Willis ring.

16. (canceled)

17. (canceled)

18. The method of claim 1, further comprising:
performing an application analysis on the blood flow parameter of the reticular vessel model.

19. A system for determining a blood flow parameter of a reticular vessel, comprising:
at least one storage device for storing computer instructions; and
at least one processor configured to communicate with the at least one storage device, wherein when executing the computer instructions, the at least one processor is configured to perform operations including:
obtaining vessel data of an object;
generating a reticular vessel model by performing, based on the vessel data, vessel segmentation;
generating, by performing model partitioning based on the reticular vessel model, a plurality of vessel sub-models; and
determining the blood flow parameter of the reticular vessel model by performing, based on the plurality of vessel sub-models, coupling processing.

20-24. (canceled)

25. The system of claim 19, wherein the determining the blood flow parameter of the reticular vessel model by performing, based on the plurality of vessel sub-models, coupling processing includes:
determining, based on physiological data of the object, a blood flow parameter of a relative pre-order sub-model of the plurality of vessel sub-models; and
determining a blood flow parameter of a relative post-order sub-model of the plurality of vessel sub-models by performing, based on a connection and the blood flow parameter of the relative pre-order sub-model.

26-27. (canceled)

28. The system of claim 25, wherein the determining a blood flow parameter of a relative post-order sub-model of the plurality of vessel sub-models by performing, based on a connection and the blood flow parameter of the relative pre-order sub-model includes:
determining, based on the connection and the blood flow parameter of the relative pre-order sub-model, a boundary parameter of the relative post-order sub-model; and
determining, based on the boundary parameter of the relative post-order sub-model, the blood flow parameter of the relative post-order sub-model.

29. The system of claim 19, wherein the determining the blood flow parameter of the reticular vessel model by performing, based on the plurality of vessel sub-models, coupling processing includes:
modifying a boundary parameter of at least one of the plurality of vessel sub-models corresponding to a collision flow; and
determining a modified blood flow parameter of the at least one of the plurality of vessel sub-models by re-performing, based on the modified boundary parameter, the coupling processing.

30-37. (canceled)

38. A non-transitory computer readable medium, comprising at least one set of instructions, wherein when executed by one or more processors of a computing device, the at least one set of instructions causes the computing device to perform a method, the method comprising:
obtaining vessel data of an object;
generating a reticular vessel model by performing, based on the vessel data, vessel segmentation;
generating, by performing model partitioning based on the reticular vessel model, a plurality of vessel sub-models; and
determining the blood flow parameter of the reticular vessel model by performing, based on the plurality of vessel sub-models, coupling processing.
